(19) United States
(12) Patent Application Publication
(10) Pub. No.: US 2003/0232013 A1
(43) Pub. Date: Dec. 18, 2003

(54) Therapeutic and diagnostic targeting of cancers cells with tumor homing peptides

(76) Inventors: Gary Sieckman, Ashland, MO (US); Wynn Volkert, Columbia, MO (US); Leonard Forte, Columbia, MO (US); Timothy Hoffman, Columbia, MO (US); Hariprasad Gali, College Station, TX (US)

Correspondence Address: Steven L. Highlander, FULBRIGHT & JAWORSKI L.L.P., SUITE 2400, 600 CONGRESS AVENUE, AUSTIN, TX, 78701-3271, US

(21) Appl. No.: 10/371,966

(22) Filed: Feb. 20, 2003

Related U.S. Application Data
(60) Provisional application No. 60/359,204, filed on Feb. 22, 2002.

(51) Int. Cl. A61K 51/00; A61K 49/00; A61K 48/00; A61K 38/19

(52) U.S. Cl. 424/1.69; 424/9.322; 424/85.1; 514/44

(57) ABSTRACT

The present invention provides methods of targeting breast cancer, prostate cancer, pancreatic cancer or melanoma cells using ST peptides. These methods permit both diagnostic evaluation and therapeutic intervention using appropriate conjugates.

This application claims benefit of priority to U.S. Provisional Serial No. 60/359,204, filed Feb. 22, 2002, the entire contents of which are hereby incorporated by reference.

The government owns rights in the present invention pursuant to grant number DOE DEFG02ER60877 from the Department of Energy.

### BACKGROUND OF THE INVENTION

I. Field of the Invention

The present invention relates generally to the fields of cell biology and oncology. More particularly, it concerns the use of tumor homing peptides to deliver therapeutic and diagnostic agents to cancer cells.

II. Description of Related Art

One of the primary challenges presented in oncology is the targeting of therapeutic and diagnostic agents to cancer tissues. A traditional approach is the use of antibodies or tumor-homing peptides that bind to targets found on the surfaces of cancer cells, but not found on normal tissues. Such targets, which must have minimal homology to other cell surface molecules, are difficult to find. The greater degree of cross-reactivity between a given target and a distinct molecule, the less accurate a diagnosis, and the more harmful a therapy will be. Furthermore, due to the heterogeneity of tumors, the absence of useful targets on some cancers limits the efficacy of the targeting means.

Waldmann and colleagues reported that colorectal cells express receptors that bind specifically to E. coli heat stable enterotoxin (ST), guanylin, human uroguanylin and ST analogues (Carrithers et al., 1994; Carrithers et al., 1996; U.S. Pat. No. 5,518,888). Thus, Waldmann believed that peptides containing the ST motif had potential utility for targeting therapeutic and diagnostic agents to colorectal cancers.

The identity of the receptor expressed on the surface of colorectal cells is not clear. There is ample evidence in the literature that the guanylin receptor on intestinal epithelial cells and colorectal cells is primarily responsible for the binding of guanylin, uroguanylin and ST peptides (Forte, 1999; Semrad, 1997). The guanylin receptors on these cells are classified as guanylate cyclase-C (GC-C) receptors (Forte, 1999). There is no indication that such receptors would be expressed on other cancer cells.

### SUMMARY OF THE INVENTION

Thus, in accordance with the present invention, there is provided a method for targeting an agent to a breast cancer cell, a prostate cancer cell, a pancreatic cancer cell or a melanoma cancer cell comprising bringing the cancer cell into contact with a peptide-agent complex, wherein the peptide comprises an ST motif that binds to breast cancer cells, prostate cancer cells, pancreatic cancer cells or melanoma cancer cells. The agent may be a diagnostic agent, such as a radiolabel, a chemilluminescent label, a fluorescent label, a magnetic spin resonance label, or a dye, or it may be a therapeutic agent, such as a chemotherapeutic agent, a radiotherapeutic agent, a toxin, a cytokine or a nucleic acid construct. The ST motif may be an STₕ motif, such as Y—Rb₍₆₋₁₈₎—X, wherein Y is a tail region comprising a linear segment of 0-10 amino acid residues, Rb₍₆₋₁₈₎ is a receptor binding region, and X it Tyr or Phe. The tail region may comprise Asn-Ser-Ser-Asn-Tyr, and Rb₍₆₋₁₈₎ may comprise Cys-Cys-Glu-Leu-Cys-Cys-Asn-Pro-Ala-Cys-Thr-Gly-Cys.

The ST motif may comprises the wild-type human ST sequence. The complex may further comprise a linking moiety that connects the agent and the peptide, such as a moiety is linked to the ST peptide through the N-terminal amine. The cancer cell may be located in a subject, for example, a human subject. The complex may be delivered local or regional to the cancer cell, or delivered systemically.

In another embodiment, there is provided a method for diagnosing breast cancer, prostate cancer, pancreatic cancer or melanoma in a subject comprising (a) administering to the subject a peptide-diagnostic agent complex, wherein the peptide comprises an ST motif, wherein the ST motif binds to breast cancer cells, prostate cancer cells, pancreatic cancer cells or melanoma cancer cells; and (b) assessing the amount and/or localization in the subject, of the diagnostic agent. The patient may or may not have been previously diagnosed with cancer. The patient may be at elevated risk for one or more of breast cancer, prostate cancer, pancreatic cancer or melanoma. The assessing may comprise organ or whole body imaging.

In yet another embodiment, there is provided a method for treating breast cancer, prostate cancer, pancreatic cancer or melanoma in a subject in need thereof comprising administering to the subject a peptide-therapeutic agent complex, wherein the peptide comprises an ST motif and binds to breast cancer cells, prostate cancer cells, pancreatic cancer cells or melanoma cancer cells. The complex administered more than once, and may be delivered local or regional to a tumor, or delivered systemically. The method may further comprise administering a second distinct cancer therapy, such as radiotherapy, chemotherapy, immunotherapy or surgery.

In still yet another embodiment, there is provided a method for rendering an unresectable breast, prostate, pancreatic or melanoma tumor resectable comprising administering to a subject having the tumor a peptide-therapeutic agent complex, wherein the peptide comprises an ST motif that binds to breast cancer cells, prostate cancer cells, pancreatic cancer cells or melanoma cancer cells.

In yet a further embodiment, there is provided a method for treating metastatic breast cancer, prostate cancer, pancreatic cancer or melanoma comprising administering to a subject in need thereof a peptide-therapeutic agent complex, wherein the peptide comprises an ST motif that binds to breast cancer cells, prostate cancer cells, pancreatic cancer cells or melanoma cancer cells.

In an additional embodiment, there is provided a method for preventing recurrent breast cancer, prostate cancer, pancreatic cancer or melanoma comprising administering to a subject having been successfully treated for breast cancer, prostate cancer, pancreatic cancer or melanoma a peptide-therapeutic agent complex, wherein the peptide comprises an ST motif that binds to breast cancer cells, prostate cancer cells, pancreatic cancer cells or melanoma cancer cells.

In still an additional embodimemt, there is provided a method for identifying tumor binding peptides comprising (a) providing a breast cancer cell, a prostate cancer cell, a pancreatic cancer cell or a melanoma cell; (b) contacting the cell, in the presence of a candidate peptide, with a labeled, tumor-binding ST peptide that binds to breast cancer cells, prostate cancer cells, pancreatic cancer cells or melanoma cancer cells; (c) measuring the association of label with the cell, as compared to the association of label with the cell in the absence of the candidate peptide; and (d) measuring binding of the candidate peptide to ST peptide, wherein a decrease in association of label with the cell, and the absence of candidate peptide binding to ST peptide, indicates that the candidate peptide is competing with ST peptide for tumor cell binding. The method may further comprise labeling the candidate peptide, incubating the labeled candidate peptide with the cell, and measuring the association of label with the cell.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1—Structure of heat-stable enterotoxin (STₕ) produced by human strain of Escherichia coli bacteria.

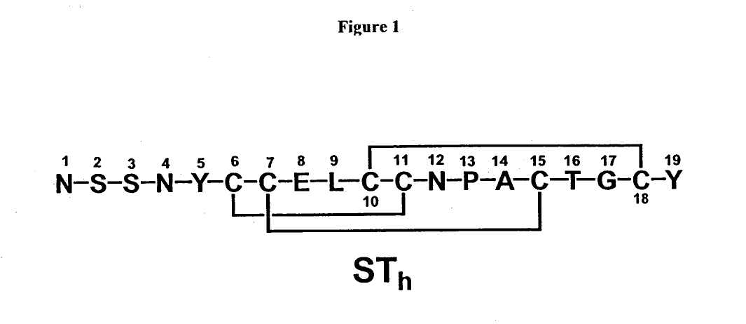

FIG. 2—Structure of Phe¹⁹-STₕ.

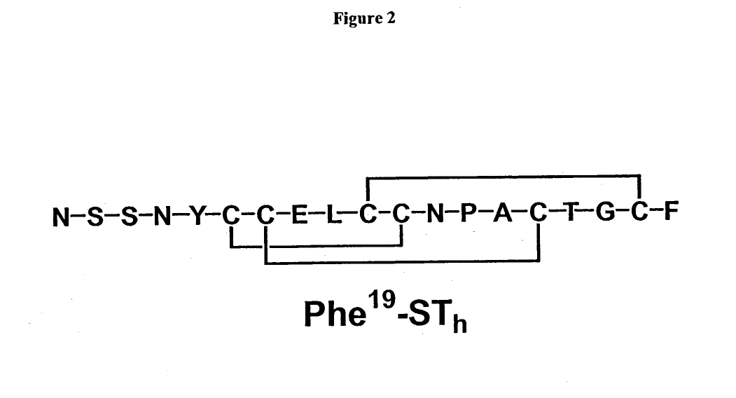

FIG. 3—General structure of STₕ analogs.

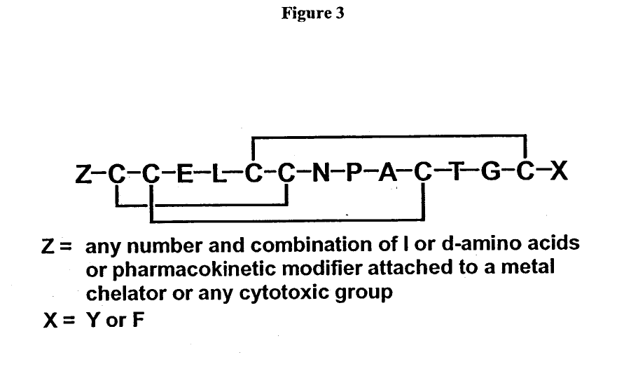

FIG. 4—Structure of DOTA-Phe¹⁹-STₕ.

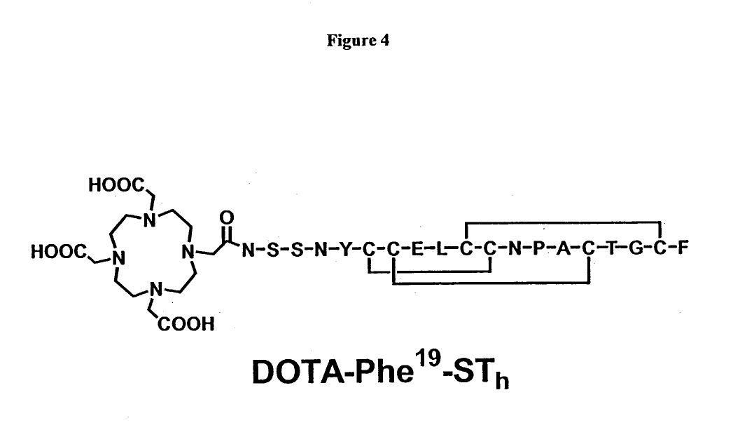

FIG. 5—Competitive binding curve of Phe¹⁹-STₕ vs ¹²⁵-Tyr-6-Ahx-Phe¹⁹-STₕ in MB-231 and T-47D cells. IC₅₀ of Phe¹⁹-STₕ is 5.2±1.3 nM for MB-231 and 3.0±1.7 nM for T-47D.

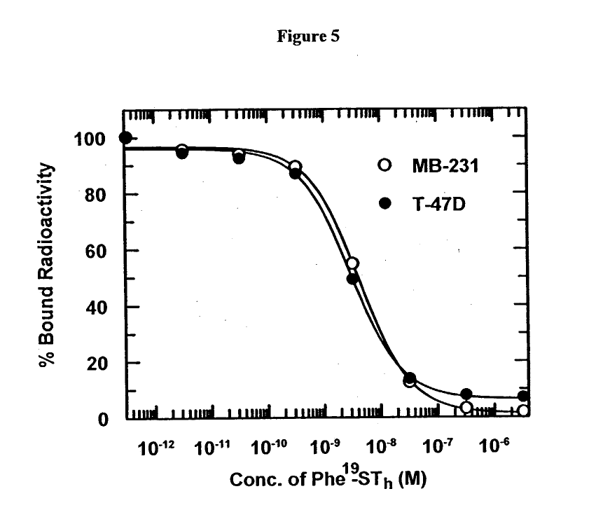

FIG. 6—Competitive binding curve of In-DOTA-Phe¹⁹-STₕ vs ¹²⁵I-Tyr⁵-Phe¹⁹-STₕ in MB-231 and T-47D cells. IC₅₀ of In-DOTA-Phe¹⁹-STₕ is 9.9±2.0 nM for MB-231 and 8.9±2.2 nM for T-47D.

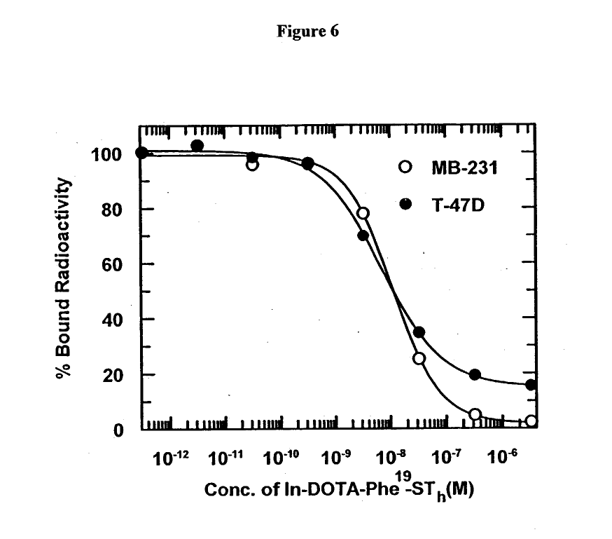

FIG. 7—Scatchard plot of ¹²⁵I-Tyr⁵-6-Ahx-Phe¹⁹-STₕ in MB-231 cells. K_{d}=4.0 nM and No. of receptors per cell at equilibrium (calculated using Bₘₐₓ value)=112,786.

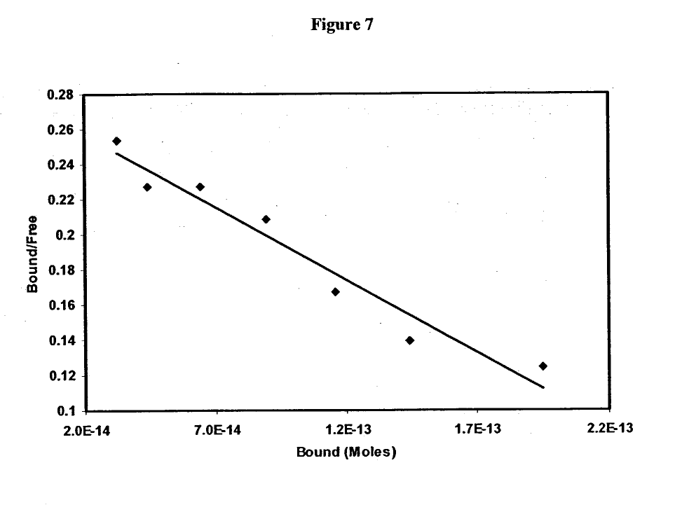

FIG. 8—Scatchard plot of ¹²⁵I-Tyr⁵-6-Ahx-Phe¹⁹-STₕ in T-47D cells. K_{d}=4.4 nM and No. of receptors per cell at equilibrium (calculated using Bₘₐₓ value)=41,758.

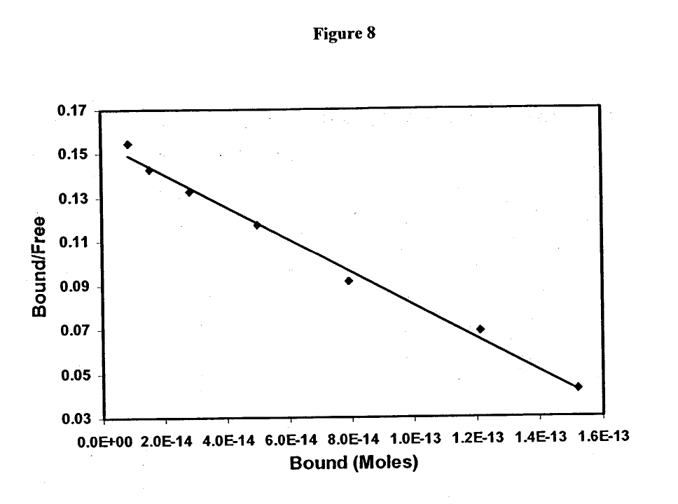

### DESCRIPTION OF ILLUSTRATIVE EMBODIMENTS

Despite tremendous advances in diagnosis and therapy, cancer continues to be a major cause of mortality in the industrialized world, and major cost center for health care. Thus, a need for new and improved methods for both identifying cancer, and for its subsequent treatment, remain of paramount importance.

I. The Present Invention

In the early 1990's, Waldman and coworkers showed that ST peptides were able to selectively bind to receptors found colon cancer cells. The proposed the use of ST peptides in targeting of diagnostic and therapeutic compounds to tumors of the colon. Surprisingly, the present inventors have discovered that ST peptides also bind to several other types of cancer, including cancers of the breast, pancreas, prostate and melanoma. Therefore, it is proposed here that the use of ST peptides in cancer diagnosis and therapy can be extended to these malignancies as well. Various aspects of the invention are discussed in the following pages.

II. ST Peptides

Heat stable enterotoxin, or “ST,” which is produced by E. coli as well as other organisms, is responsible for endemic diarrhea in developing countries and travelers diarrhea. ST induces intestinal secretion by binding to specific receptors, ST receptors, in the apical brush border membranes of the mucosal cells lining the intestinal tract. Binding of ST to ST receptors is non-covalent and occurs in a concentration-dependent and saturable fashion. Once bound, ST/ST receptor complexes appear to transported from the surface into the interior of the cell. Binding of ST to ST receptors triggers a cascade of biochemical reactions in the apical membrane of these cells resulting in the production of a signal which induces intestinal cells to secrete fluids and electrolytes, resulting in diarrhea.

ST receptors are unique in that they are only localized in the apical brush border membranes of the cells lining the intestinal tract. They are not found in any other cell type in placental mammals. In addition, ST receptors are almost exclusively localized to the apical membranes, with little being found in the basolateral membranes on the sides of intestinal cells.

Mucosal cells lining the intestine are joined together by tight junctions which form a barrier against the passage of intestinal contents into the blood stream and components of the blood stream into the intestinal lumen. Therefore, the apical location of ST receptors isolates these receptors from the circulatory system. Compositions administered “outside” the intestinal tract are maintained apart and segregated from the only cells which normally express ST receptors.

As discussed in U.S. Pat. No. 5,518,888, there are a number of distinct ST peptides, and variants thereof, all of which can be used in accordance with the present invention. In particular embodiments, the present invention will involve the ST peptides from various organisms, including human. While it is believed that some variation in the particular ST motif may be tolerated, a basic core structure required for functionality may be represented by the sequence Ser-Ser-Asn. Further delineation of this motif includes Ser-Ser-Asn-X, where X can be Phe or Tyr, giving Phe-Ser-Ser-Asn-(optionally X), and Asn-Ser-Ser-Asn-(optionally X).

1. Peptide Synthesis

While ST peptides may be isolated from natural sources using standard techniques, it will be advantageous to produce ST peptides using the solid-phase synthetic techniques (Merrifield, 1963). Other peptide synthesis techniques are well known to those of skill in the art (Bodanszky et al., 1976;) Peptide Synthesis, 1985; Solid Phase Peptide Synthelia, 1984); The Proteins, 1976. Appropriate protective groups for use in such syntheses will be found in the above texts, as well as in Protective Groups in Organic Chemistry, 1973. These synthetic methods involve the sequential addition of one or more amino acid residues or suitable protected amino acid residues to a growing peptide chain. Normally, either the amino or carboxyl group of the first amino acid residue is protected by a suitable, selectively removable protecting group. A different, selectively removable protecting group is utilized for amino acids containing a reactive side group, such as lysine.

Using solid phase synthesis as an example, the protected or derivatized amino acid is attached to an inert solid support through its unprotected carboxyl or amino group. The protecting group of the amino or carboxyl group is then selectively removed and the next amino acid in the sequence having the complementary (amino or carboxyl) group suitably protected is admixed and reacted with the residue already attached to the solid support. The protecting group of the amino or carboxyl group is then removed from this newly added amino acid residue, and the next amino acid (suitably protected) is then added, and so forth. After all the desired amino acids have been linked in the proper sequence, any remaining terminal and side group protecting groups (and solid support) are removed sequentially or concurrently, to provide the final peptide. The peptides of the invention are preferably devoid of benzylated or methylbenzylated amino acids. Such protecting group moieties may be used in the course of synthesis, but they are removed before the peptides are used. Additional reactions may be necessary, as described elsewhere, to form intramolecular linkages to restrain conformation.

2. Peptide Conjugation

Bifunctional cross-linking reagents have been extensively used for a variety of purposes including preparation of affinity matrices, modification and stabilization of diverse structures, identification of ligand and receptor binding sites, and structural studies. Homobifunctional reagents that carry two identical functional groups proved to be highly efficient in inducing cross-linking between identical and different macromolecules or subunits of a macromolecule, and linking of polypeptide ligands to their specific binding sites. Heterobifunctional reagents contain two different functional groups. By taking advantage of the differential reactivities of the two different functional groups, cross-linking can be controlled both selectively and sequentially. The bifunctional cross-linking reagents can be divided according to the specificity of their functional groups, e.g., amino, sulfhydryl, guanidino, indole, carboxyl specific groups. Of these, reagents directed to free amino groups have become especially popular because of their commercial availability, ease of synthesis and the mild reaction conditions under which they can be applied. A majority of heterobifunctional cross-linking reagents contains a primary amine-reactive group and a thiol-reactive group.

Exemplary methods for cross-linking ligands to liposomes are described in U.S. Pat. Nos. 5,603,872 and 5,401,511, each specifically incorporated herein by reference in its entirety). Various agents can be covalently bound to ST peptides through the cross-linking of amine residues. Liposomes (see below), in particular, multilamellar vesicles (MLV) or unilamellar vesicles such as microemulsified liposomes (MEL) and large unilamellar liposomes (LUVET), each containing phosphatidylethanolamine (PE), have linked by established procedures. The inclusion of PE in the liposome provides an active functional residue, a primary amine, on the liposomal surface for cross-linking purposes. ST peptides are bound covalently to discrete sites on the liposome surfaces. The number and surface density of these sites will be dictated by the liposome formulation and the liposome type. The liposomal surfaces may also have sites for non-covalent association. To form covalent conjugates of ST peptides and liposomes, cross-linking reagents have been studied for effectiveness and biocompatibility. Cross-linking reagents include glutaraldehyde (GAD), bifunctional oxirane (OXR), ethylene glycol diglycidyl ether (EGDE), and a water soluble carbodiimide, preferably 1-ethyl-3-(3-dimethylaminopropyl) carbodiimide (EDC). Through the complex chemistry of cross-linking, linkage of the amine residues of the recognizing substance and liposomes is established.

In another example, heterobifunctional cross-linking reagents and methods of using the cross-linking reagents are described in U.S. Pat. No. 5,889,155, specifically incorporated herein by reference in its entirety. The cross-linking reagents combine a nucleophilic hydrazide residue with an electrophilic maleimide residue, allowing coupling in one example, of aldehydes to free thiols. The cross-linking reagent can be modified to cross-link various functional groups and is thus useful for cross-linking polypeptides. Table 1 details certain hetero-bifunctional cross-linkers considered useful in the present invention

TABLE 1
HETERO-BIFUNCTIONAL CROSS-LINKERS

| | | | Spacer Arm Length/ | |
|---|---|---|---|---|
| Linker | Reactive Toward | Advantages and Applications | after cross-linking | |
| SMPT | Primary amines Sulfhydryls | Greater stability | 11.2 | A |
| SPDP | Primary amines Sulfhydryls | Thiolation | 6.8 | A |
| | | Cleavable cross-linking | | |
| LC-SPDP | Primary amines Sulfhydryls | Extended spacer arm | 15.6 | A |
| Sulfo-LC-SPDP | Primary amines Sulfhydryls | Extended spacer arm | 15.6 | A |
| | | Water-soluble | | |
| SMCC | Primary amines Sulfhydryls | Stable maleimide reactive group | 11.6 | A |
| | | Enzyme-antibody conjugation | | |
| | | Hapten-carrier protein conjugation | | |
| Sulfo-SMCC | Primary amines Sulfhydryls | Stable maleimide reactive group | 11.6 | A |
| | | Water-soluble | | |
| | | Enzyme-antibody conjugation | | |
| MBS | Primary amines Sulfhydryls | Enzyme-antibody conjugation | 9.9 | A |
| | | Hapten-carrier protein conjugation | | |
| Sulfo-MBS | Primary amines Sulfhydryls | Water-soluble | 9.9 | A |
| SIAB | Primary amines Sulfhydryls | Enzyme-antibody conjugation | 10.6 | A |
| Sulfo-SIAB | Primary amines Sulfhydryls | Water-soluble | 10.6 | A |
| SMPB | Primary amines Sulfhydryls | Extended spacer arm | 14.5 | A |
| | | Enzyme-antibody conjugation | | |
| Sulfo-SMPB | Primary amines Sulfhydryls | Extended spacer arm | 14.5 | A |
| | | Water-soluble | | |
| EDC/Sulfo- | Primary amines Carboxyl | Hapten-Carrier conjugation | 0 | |
| NHS | groups | | | |
| ABH | Carbohydrates Nonselective | Reacts with sugar groups | 11.9 | A |

In instances where a particular ST peptide does not contain a residue amenable for a given cross-linking reagent in its native sequence, conservative genetic or synthetic amino acid changes in the primary sequence can be utilized.

III. Cancers

In accordance with the present invention, it has been determined that several cancer, in addition to colorectal cancer, express receptors for ST peptides. The present application provide data showing that ST peptides bind with high affinity and specificity to breast cancer cells, prostate cancer cells, pancreatic cancer cells and melanoma cells.

Thus, each of these cancers are suitable targets for ST-based diagnostic and therapeutic methods.

1. Breast Cancer

Other than skin cancer, breast cancer is the most common type of cancer among women in the United States. More than 180,000 women are diagnosed with breast cancer each year. The National Cancer Institute (NCI) has written this booklet to help patients with breast cancer and their families and friends better understand this disease. We hope others will read it as well to learn more about breast cancer.

The most common type of breast cancer is ductal carcinoma. It begins in the lining of the ducts. Another type, called lobular carcinoma, arises in the lobules. When cancer is found, the pathologist can tell what kind of cancer it is (whether it began in a duct or a lobule) and whether it is invasive. Depending on the extent of the cancer, it is graded from carcinoma in situ, and Stages I-IV, from least to most serious.

Therapy almost always involves some form of surgery, either a “lumpectomy” that is designed to remove only the tumor, or partial or segmental mastectomy, which results in the loss of significant breast tissue. Peripheral lymph nodes also may be removed. Radical mastectomy, which involves removal of underlying chest muscle, is only used when the cancer has spread to that tissue. Other common treatments include radiation therapy, chemotherapy (often taxol), and hormone therapy (estrogen or progesterone). Immunotherapy, bone marrow transplantation, and peripheral blood stem cell transplantation are more experimental options.

2. Prostate Cancer

The prostate is a gland found in all men. It is about the size of a walnut, and is located below the bladder and in front of the rectum. The urethra, the tube that drains the bladder, passes through the prostate and into the penis. The primary function of the prostate is to produce fluid that helps carry sperm from the testicles. It thus serves a function in reproduction.

Prostate cancer is a result of genetic and environmental changes that cause glandular cells in the prostate to multiply abnormally. In addition to causing problems within the prostate, they can spread to other organs as well, severely complicating treatment. Prostate cancer has other characteristics as well. Cancer in the prostate is usually a very slow disease to progress compared with cancers in other organs. It is not unusual, however, for a person to have no symptoms or signs of the disease that would be recognized without a doctor's involvement.

Unfortunately, cancer of the prostate is a very common cancer. At 50, one-third of all men have microscopic evidence of prostate cancer, and at 75, half to three-quarters of all men will have prostate cancer. Most prostate cancers can be categorized as being latent, showing no clinical signs or symptoms, or indolent, meaning they are growing so slowly that they pose little health threat. Nonetheless, about 180,000 men are diagnosed with prostate cancer each year, and close to 85% of these men would benefit from treatment. About 40,000 men die each year from this form of cancer.

3. Pancreatic Cancer

Pancreatic cancer occurs when a malignant tumor(s) forms in the pancreas, an elongated gland located deep in the abdomen that facilitates digestion and the regulation of blood sugars. An extremely aggressive malignancy, pancreatic cancer is the fourth leading cause of cancer deaths among U.S. men, and the fifth leading cause in women. Nationwide, some 27,000 new cases are diagnosed annually. Close to 30,000 deaths are attributed to the disease each year. Unfortunately, by the time pancreatic cancer is diagnosed, it is usually too late for a promising outcome. The average life expectancy after being diagnosed with pancreatic cancer is 3 to 6 months.

Although the exact cause of pancreatic cancer remains unknown, several risk factors have been identified. In addition to advanced age (most cases occur between the ages of 65 and 79), smoking is a primary risk factor (incidence rates are more than twice as high for smokers than nonsmokers). Excessive dietary fat also may promote the disease, and some studies have shown a link between pancreatic cancer and chronic pancreatitis, diabetes, or cirrhosis. Certain industrial compounds also have been linked to increases in pancreatic cancer.

Pancreatic cancer usually does not produce symptoms until it has reached an advanced stage. Such symptoms may include significant weight loss accompanied by abdominal pain; persistent back pain that worsens when eating or lying down; digestive or bowel problems such as light-colored stools, diarrhea, bloating or gas; dark-colored urine; nausea, vomiting or loss of appetite; occurrence of jaundice, a yellowish discoloration of the skin and whites of the eyes; and sudden onset of diabetes. Because these symptoms may be confused with other disease, delayed diagnosis is quite possible, which leads to fatal results.

4. Melanoma

Melanoma is a serious form of skin cancer. It begins in melanocytes, which are cells that make the skin pigment melanin. Although melanoma accounts for only about 4% of all skin cancer cases, it is the cause of most skin cancer-related deaths. The good news is that melanoma is often curable if it is detected and treated in its early stages. In men, melanoma is found most often on the area between the shoulders and hips or on the head and neck. In women, melanoma often develops on the lower legs. It may also appear under the fingernails or toenails or on the palms or soles. The chance of developing melanoma increases with age, but it affects all age groups and is one of the most common cancers in young adults.

The number of new melanomas diagnosed in the United States is increasing. Since 1973, the rate of new melanomas diagnosed per year has more than doubled from 6 per 100,000 to 14 per 100,000. The American Cancer Society estimates that about 51,400 new melanomas will be diagnosed in the United States during 2001. About 7,800 cancer deaths will be attributed to malignant melanoma in 2001.

When melanoma starts in the skin, it is called cutaneous melanoma. Melanoma may also occur in the eye (ocular melanoma or intraocular melanoma) and, rarely, in other areas where melanocytes are found, such as the digestive tract, meninges, or lymph nodes. When melanoma metastasizes, cancer cells are also found in the lymph nodes and possibly also other parts of the body, such as the liver, lungs, or brain. In these cases, the cancer cells are still melanoma cells, and the disease is called metastatic melanoma.

IV. Diagnostic Agents and Methods

In accordance with the present invention, there are provided diagnostic methods for detecting cancer cells. Many appropriate imaging agents are known in the art, as are methods for their attachment to antibodies (see, for e.g., U.S. Pat. Nos. 5,021,236; 4,938,948; and 4,472,509, each incorporated herein by reference). The imaging moieties used can be paramagnetic ions; radioactive isotopes; fluorochromes; NMR-detectable substances; X-ray imaging.

In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and/or erbium (III), with gadolinium being particularly preferred. Ions useful in other contexts, such as X-ray imaging, include but are not limited to lanthanum (III), gold (III), lead (II), and especially bismuth (III).

In the case of radioactive isotopes for therapeutic and/or diagnostic application, one might mention astatine²¹¹, ⁵¹chromium, ³⁶chlorine, ⁵⁷cobalt, ⁵⁸cobalt, copper⁶⁷, ¹⁵²europium, gallium⁶⁷, iodine¹²³, iodine¹²⁵, iodine¹³¹, indium¹¹¹, ⁵⁹⁻iron, ³²phosphorus, rhenium¹⁸⁶, rhenium¹⁸⁸, ⁷⁵selenium, ³⁵sulphur, technicium^{99m} and/or yttrium⁹⁰. Of particular interest are lutetium¹⁷⁷, samarium¹⁵³, holmium¹⁶⁶ and actinium²²⁵. Also, see Table 2, below. Radioactively labeled ST peptides of the present invention may be produced according to well-known methods in the art. For instance, monoclonal antibodies can be iodinated by contact with sodium and/or potassium iodide and a chemical oxidizing agent such as sodium hypochlorite, or an enzymatic oxidizing agent, such as lactoperoxidase. ST peptides according to the invention may be labeled with technetium^{99m} by ligand exchange process, for example, by reducing pertechnate with stannous solution, chelating the reduced technetium onto a Sephadex column and applying the antibody to this column. Alternatively, direct labeling techniques may be used, e.g., by incubating pertechnate, a reducing agent such as SNCl₂, a buffer solution such as sodium-potassium phthalate solution, and the antibody. Intermediary functional groups which are often used to bind radioisotopes which exist as metallic ions to antibody are diethylenetriaminepentaacetic acid (DTPA) or ethylene diaminetetracetic acid (EDTA).

Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

V. Therapeutic Agents

The present invention also provides for the delivery of therapeutic agents to cancer cells using ST peptides to target such agents. The agents may be linked directly to the peptide (above), or they may be encapsulated in a liposome (below) which, in turn, is targeted by the ST peptide. Some examples of therapeutic agents are discussed in the following pages.

1. Radiopharmaceuticals

A number of different radioactive substances can be used in cancer therapy. Examples of radioactive isotopes for therapeutic applications include astatine²¹¹, ⁵¹chromium, ³⁶chlorine, ⁵⁷cobalt, ⁵⁸cobalt, copper⁶⁷, ¹⁵²europium, gallium⁶⁷, iodine¹²³, iodine¹²⁵, iodine¹³¹, indium¹¹¹, ⁵⁹⁻iron, ³²phosphorus, rhenium¹⁸⁶, rhenium¹⁸⁸, ⁷⁵selenium, ³⁵sulphur, technicium^{99m}, yttrium⁹⁰, lutetium¹⁷⁷, samarium¹⁵³, holmium¹⁶⁶, and actinium²²⁵. Also, see Table 2, below.

TABLE 2
THERAPEUTIC AND DIAGNOSTIC RADIOACTIVE ISOTOPES

| Isotope | Half-Life | Indication |
|---|---|---|
| Ac-225 | 10.0d | Monoclonal antibody attachment used for cancer treatment |
| | | (RIT), also parent of Bi-213. |
| Ac-227 | 21.8y | Parent of Ra-223 (Monoclonal antibody attachment used |
| | | for cancer treatment (RIT). |
| Am-241 | 432y | Osteoporosis detection, heart imaging. |
| As-72 | 26.0h | Planar imaging, SPECT or PET. |
| As-74 | 17.8d | Positron-emitting isotope with biomedical applications. |
| At-211 | 7.21h | Monoclonal antibody attachment (alpha emitter) used for |
| | | cancer treatment (RIT), used with F-18 for in vivo studies. |
| Au-198 | 2.69d | Cancer treatment using mini-gun (B), treating ovarian, |
| | | prostate, and brain cancer. |
| B-11 | Stable | Melanoma and brain tumor treatment. |
| Be-7 | 53.2d | Used in berylliosis studies. |
| Bi-212 | 1.10h | Monoclonal antibody attachment (alpha emitter) used for |
| | | cancer treatment (RIT), cellular dosimetry studies. |
| Bi-213 | 45.6m | Monoclonal antibody attachment (alpha emitter) used for |
| | | cancer treatment (RIT). |
| Br-75 | 98m | Planar imaging, SPECT or PET (C). |
| Br-77 | 57h | Label radiosentizers for Te quantization of hypoxia in |
| | | tumors, and monoclonal antibody labeling. |
| C-11 | 20.3m | Radiotracer in PET scans to study normal/abnormal brain |
| | | functions. |
| C-14 | 5730y | Radiolabeling for detection of tumors (breast and others). |
| Ca-48 | Stable | |
| Cd-109 | 462d | Cancer detection (C), pediatric imaging (C). |
| Ce-139 | 138d | Calibrates high-purity germanium gamma detectors. |
| Ce-141 | 32.5d | Gastrointestinal tract diagnosis, measuring regional |
| | | myocardial blood flow. |
| Cf-252 | 2.64y | Cervical, melanoma, brain cancer treatment. |
| Co-55 | 17.5h | Planar imaging, SPECT or PET (B). Used in PET imaging |
| | | of damaged brain tissue after stroke. |
| Co-57 | 272d | Gamma camera calibration, should be given high priority, |
| | | radiotracer in research and a source for X-ray fluorescence |
| | | spectroscopy. |
| Co-60 | 5.27y | Teletherapy (destroy cancer cells), disinfect surgical |
| | | equipment and medicines, external radiation cancer |
| | | therapy (E). |
| Cr-51 | 27.7d | Medical, cell labeling and dosimetry. |
| Cs-130 | 29.2m | Myocardial localizing agent. |
| Cs-131 | 9.69d | Intracavity implants for radiotherapy. |
| Cs-137 | 30.2y | Blood irradiators, PET imaging, tumor treatment. |
| Cu-61 | 3.35h | Planar imaging, SPECT or PET (B). |
| Cu-62 | 4.7m | Positron emitting radionuclide (B), cerebral and myocardial |
| | | blood flow used As-a tracer in conjunction with Cu 64 (B). |
| Cu-64 | 12.7h | PET scanning (C), planar imaging (C), SPECT imaging (C) |
| | | dosimetry studies (C), cerebral and myocardial blood flow |
| | | (C), used with Cu-62 (C), treating of colorectal cancer. |
| Cu-67 | 61.9h | Cancer treatment/diagnostics, monoclonal antibodies, |
| | | radioimmunotherapy, planar imaging, SPECT or PET. |
| Dy-165 | 2.33h | Radiation synovectomy, rheumatoid arthritis treatment. |
| Eu-152 | 13.4y | Medical. |
| Eu-155 | 4.73y | Osteoporosis detection. |
| F-18 | 110m | Radiotracer for brain studies (C), PET imaging (C). |
| Fe-55 | 2.73y | Heat source. |
| Fe-59 | 44.5d | Medical. |
| Ga-64 | 2.63m | Treatment of pulmonary diseases ending in fibrosis of |
| | | lungs. |
| Ga-67 | 78.3h | Imaging of abdominal infections (C), detect Hodgkins/non- |
| | | Hodgkins lymphoma (C), used with In-111 for soft tissue |
| | | infections and osteomyelitis detection (C), evaluate |
| | | sarcoidiodis and other granulomaous diseases, particularly |
| | | in lungs and mediastiusim (C). |
| Ga-68 | 68.1m | Study thrombosis and atherosclerosis, PET imaging, |
| | | detection of pancreatic cancer, attenuation correction. |
| Gd-153 | 242d | Dual photon source, osteoporosis detection, SPECT |
| | | imaging. |
| Ge-68 | 271d | PET imaging. |
| H-3 | 12.3y | Labeling, PET imaging. |
| I-122 | 3.6m | Brain blood flow studies. |
| I-123 | 13.1h | Brain, thyroid, kidney, and myocardial imaging (C), |
| | | cerebral blood flow (ideal for imaging) (C), neurological |
| | | disease (Alzheimer's) (C). |
| I-124 | 4.17d | Radiotracer used to create images of human thyroid, PET |
| | | imaging. |
| I-125 | 59.9d | Osteoporosis detection, diagnostic imaging, tracer for |
| | | drugs, monoclonal antibodies, brain cancer treatment (I-131 |
| | | replacement), SPECT imaging, radiolabeling, tumor |
| | | imaging, mapping of receptors in the brain (A), interstitial |
| | | radiation therapy (brachytherapy) for treatment of prostate |
| | | cancer (E). |
| I-131 | 8.04d | Lymphoid tissue tumor/hyperthyroidism treatment (C), |
| | | antibody labeling (C), brain biochemistry in mental illness |
| | | (C), kidney agent (C), thyroid problems (C), alternative to |
| | | T1-201 for radioimmunotherapy (C), imaging, cellular |
| | | dosimetry, scintigraphy, treatment of graves disease, |
| | | treatment of goiters, SPECT imaging, treatment of prostate |
| | | cancer, treatment of hepatocellular carcinoma, treatment |
| | | of melanoma (A), locate osteomyelitis infections (A), |
| | | radiolabeling (A), localize tumors for removal (A), |
| | | treatment of spinal tumor (A), locate metastatic lesions (A), |
| | | treat-neuroblastoma (A), internal (systemic) radiation |
| | | therapy (E), treatment of carcinoma of the thyroid (E). |
| I-132 | 2.28h | Mapping precise area of brain tumor before operating. |
| In-111 | 2.81d | Detection of heart transplant rejection (C), imaging of |
| | | abdominal infections (C), antibody labeling (C) cellular |
| | | immunology (C), used with Ga-67 for soft tissue infection |
| | | detection and ostemyelitis detection (C), concentrates in |
| | | liver, kidneys (C), high specific activity (C), white blood |
| | | cell imaging, cellular dosimetry, myocardial scans, |
| | | treatment of leukemia, imaging tumors. |
| In-115m | 4.49h | Label blood elements for evaluating inflammatory bowel |
| | | disease. |
| Ir-191m | 6s | Cardiovascular angiography. |
| Ir-192 | 73.8d | Implants or “seeds” for treatment of cancers of the prostate, |
| | | brain, breast, gynecological cancers. |
| Kr-81m | 13.3s | Lung imaging. |
| Lu-177 | 6.68d | Heart disease treatment (restenosis therapy), cancer |
| | | therapy. |
| Mn-51 | 46.2m | Myocardial localizing agent. |
| Mn-52 | 5.59d | PET scanning. |
| Mo-99 | 65.9h | Parent for Tc-99m generator used for brain, liver, lungs, |
| | | heart imaging. |
| N-13 | 9.97m | PET imaging, myocardial perfusion. |
| Nb-95 | 35d | Study effects of radioactivity on pregnant women and fetus, |
| | | myocardial tracer, PET imaging. |
| O-15 | 122s | Water used for tomographic measuring of cerebral blood |
| | | flow (C), PET imaging (C), SPECT imaging. |
| Os-191 | 15.4d | Parent for Ir-191m generator used for cardiovascular |
| | | angiography. |
| Os-194 | 6.00y | Monoclonal antibody attachment used for cancer treatment |
| | | (RIT). |
| P-32 | 14.3d | Polycythaemia Rubra Vera (blood cell disease) and |
| | | leukemia treatment, bone disease diagnosis/treatment, |
| | | SPECT imaging of tumors (A), pancreatic cancer treatment |
| | | (A), radiolabeling (A). |
| P-33 | 25d | Labeling. |
| Pb-203 | 2.16d | Planar imaging, SPECT or PET (used with Bi-212) (B), |
| | | monoclonal antibody immunotherapy (B), cellular |
| | | dosimetry. |
| Pb-212 | 10.6h | Radioactive label for therapy using antibodies, cellular |
| | | dosimetry. |
| Pd-103 | 17d | Prostate cancer treatment. |
| Pd-109 | 13.4h | Potential radiotherapeutic agent. |
| Pu-238 | 2.3y | Pacemaker (no Pu-236 contaminants). |
| Ra-223 | 11.4d | Monoclonal antibody attachment (alpha emitter) used for |
| | | cancer treatment (RIT). |
| Ra-226 | 1.60e3y | Target isotope to make Ac-227, Th-228, Th-229 (Parents of |
| | | alpha emitters used for RIT). |
| Rb-82 | 1.27m | Myocardial imaging agent, early detection of coronary |
| | | artery disease, PET imaging, blood flow tracers. |
| Re-186 | 3.9d | Cancer treatment/diagnostics, monoclonal antibodies, bone |
| | | cancer pain relief, treatment of rheumatoid arthritis, |
| | | treatment of prostate cancer, treating bone pain. |
| Re-188 | 17h | Monoclonal antibodies, cancer treatment. |
| Rh-105 | 35.4h | Potential therapeutic applications: target neoplastic cells |
| | | (e.g., small cell lung cancer) (A), labeling of molecules and |
| | | monoclonal antibodies (A). |
| Ru-97 | 2.89d | Monoclonal antibodies label (C), planar imaging (C), |
| | | SPECT or PET techniques (C), gamma-camera imaging. |
| Ru-103 | 39d | Myocardial blood flow, radiolabeling mircospheres, PET |
| | | imaging. |
| S-35 | 87.2d | Nucleic acid labeling, P-32 replacement, cellular |
| | | dosimetry. |
| Sc-46 | 84d | Regional blood flow studies, PET imaging. |
| Sc-47 | 3.34d | Cancer treatment/diagnostics (F), monoclonal antibodies |
| | | (F), radioimmunotherapy (F). |
| Se-72 | 8.4d | Brain imaging, generator system with As-72, monoclonal |
| | | antibody immunotherapy. |
| Se-75 | 120d | Radiotracer used in brain studies, scintigraphy scanning. |
| Si-28 | Stable | Radiation therapy of cancer. |
| Sm-145 | 340d | Brain cancer treatment using I-127 (D). |
| Sm-153 | 2.00d | Cancer treatment/diagnostics (C), monoclonal antibodies |
| | | (C), bone cancer pain relief (C), higher uptake in diseased |
| | | bone than Re-186 (C), treatment of leukemia. |
| Sn-117m | 13.6d | Bone cancer pain relief. |
| Sr-85 | 65.0d | Detection of focal bone lesions, brain scans. |
| Sr-89 | 50d | Bone cancer pain palliation (improves the quality of life), |
| | | cellular dosimetry, treatment of prostate cancer, treatment |
| | | of multiple myeloma, osteoblastic therapy, potential agent |
| | | for treatment of bone metastases from prostate and breast |
| | | cancer (E). |
| Sr-90 | 29.1y | Generator system with Y-90 (B), monoclonal antibody |
| | | immunotherapy (B). |
| Ta-178 | 9.3m | Radionuclide injected into patients to allow viewing of |
| | | heart and blood vessels. |
| Ta-179 | 1.8y | X-ray fluorescence source and in thickness gauging (might |
| | | be a good substitute for Am-241). |
| Ta-182 | 115d | Bladder cancer treatment, internal implants. |
| Tb-149 | 4.13h | Monoclonal antibody attachment used for cancer treatment |
| | | (RIT). |
| Tc-96 | 4.3d | Animal studies with Tc-99m. |
| Tc-99m | 6.01h | Brain, heart, liver (gastoenterology), lungs, bones, thyroid, |
| | | and kidney imaging (C), regional cerebral blood flow (C), |
| | | equine nuclear imaging (C), antibodies (C), red blood cells |
| | | (C), replacement for Tl-201 (C). |
| Th-228 | 720d | Cancer treatment, monoclonal antibodies, parent of Bi-212. |
| Th-229 | 7300y | Grandparent for alpha emitter (Bi-213) used for cancer |
| | | treatment (RIT), parent of Ac-225. |
| Tl-201 | 73.1h | Clinical cardiology (C), heart imaging (C), less desirable |
| | | nuclear characteristics than Tc-99m for planar and SPECT |
| | | imaging (C), myocardial perfusion, cellular dosimetry. |
| Tm-170 | 129d | Portable blood irradiations for leukemia, lymphoma |
| | | treatment, power source. |
| Tm-171 | 1.9y | Medical. |
| W-188 | 69.4d | Cancer treatment, monoclonal antibodies, parent for Re- |
| | | 188 generator. |
| Xe-127 | 36.4d | Neuroimaging for brain disorders, research for variety of |
| | | neuropsychiatric disorders, especially schizophrenia and |
| | | dementia, higher resolution SPECT studies with lower |
| | | patient dose, lung imaging (some experts believe it is |
| | | superior to Xe-133 in inhalation lung studies). |
| Xe-133 | 5.25d | Lung imaging (C), regional cerebral blood flow (C), liver |
| | | imaging (gas inhalation) (C), SPECT imaging of brain, |
| | | lung scanning, lesion detection. |
| Y-88 | 107d | Substituted for Y-90 in development of cancer tumor |
| | | therapy. |
| Y-90 | 64h | Internal radiation therapy of liver cancer (C), monoclonal |
| | | antibodies (C), Hodgkins disease, and hepatoma (C), |
| | | cellular dosimetry, treating rheumatoid arthritis, treating |
| | | breast cancer, treatment of gastrointestinal |
| | | adenocarcinomas (A). |
| Y-91 | 58.5d | Cancer treatment (RIT), cellular dosimetry. |
| Yb-169 | 32d | Gastrointestinal tract diagnosis. |
| Zn-62 | 9.22h | Parent of Cu-62, a positron-emitter, used for the study of |
| | | cerebral and myocardial blood flow. |
| Zn-65 | 244d | Medical. |
| Zr-95 | 64.0d | Medical. |

A = June 1996 SNM Abstracts
B = Holmes 91
C = Herac 89
D = Fairchild 87
E = Everyone's Guide to Cancer Therapy (Dollinger, Rosenbaum, Cable), 1991
F = SNM (Society of Nuclear Medicine)

2. Chemopharmaceuticals

The term “chemotherapy” refers to the use of drugs to treat cancer. A “chemotherapeutic agent” is used to connote a compound or composition that is administered in the treatment of cancer. One subtype of chemotherapy known as biochemotherapy involves the combination of a chemotherapy with a biological therapy.

Chemotherapeutic agents include, but are not limited to, 5-fluorouracil, bleomycin, busulfan, camptothecin, carboplatin, chlorambucil, cisplatin (CDDP), cyclophosphamide, dactinomycin, daunorubicin, doxorubicin, estrogen receptor binding agents, etoposide (VP16), farnesyl-protein transferase inhibitors, gemcitabine, ifosfamide, mechlorethamine, melphalan, mitomycin, navelbine, nitrosurea, plicomycin, procarbazine, raloxifene, tamoxifen, taxol, temazolomide (an aqueous form of DTIC), transplatinum, vinblastine and methotrexate, vincristine, or any analog or derivative variant of the foregoing. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, corticosteroid hormones, mitotic inhibitors, and nitrosoureas, hormone agents, miscellaneous agents, and any analog or derivative variant thereof.

Chemotherapeutic agents and methods of administration, dosages, etc. are well known to those of skill in the art (see for example, the Goodman & Gilman's “The Pharmacological Basis of Therapeutics” and in “Remington's Pharmaceutical Sciences”, incorporated herein by reference in relevant parts), and may be combined with the invention in light of the disclosures herein. Some variation in dosage will necessarily occur depending on the condition of the subject being treated. The person responsible for administration will, in any event, determine the appropriate dose for the individual subject. Examples of specific chemotherapeutic agents and dose regimes are also described herein. Of course, all of these dosages and agents described herein are exemplary rather than limiting, and other doses or agents may be used by a skilled artisan for a specific patient or application. Any dosage in-between these points, or range derivable therein is also expected to be of use in the invention.

a. Alkylating Agents

Alkylating agents are drugs that directly interact with genomic DNA to prevent the cancer cell from proliferating. This category of chemotherapeutic drugs represents agents that affect all phases of the cell cycle, that is, they are not phase-specific. Alkylating agents can be implemented to treat, for example, chronic leukemia, non-Hodgkin's lymphoma, Hodgkin's disease, multiple myeloma, and particular cancers of the breast, lung, and ovary. An alkylating agent, may include, but is not limited to, a nitrogen mustard, an ethylenimene, a methylmelamine, an alkyl sulfonate, a nitrosourea or a triazines.

They include but are not limited to: busulfan, chlorambucil, cisplatin, cyclophosphamide (cytoxan), dacarbazine, ifosfamide, mechlorethamine (mustargen), and melphalan. In specific aspects, troglitazaone can be used to treat cancer in combination with any one or more of these alkylating agents, some of which are discussed below.

i. Nitrogen Mustards

A nitrogen mustard may be, but is not limited to, mechlorethamine (HN₂), which is used for Hodgkin's disease and non-Hodgkin's lymphomas; cyclophosphamide and/or ifosfamide, which are used in treating such cancers as acute or chronic lymphocytic leukemias, Hodgkin's disease, non-Hodgkin's lymphomas, multiple myeloma, neuroblastoma, breast, ovary, lung, Wilm's tumor, cervix testis and soft tissue sarcomas; melphalan (L-sarcolysin), which has been used to treat such cancers as multiple myeloma, breast and ovary; and chlorambucil, which has been used to treat diseases such as, for example, chronic lymphatic (lymphocytic) leukemia, malignant lymphomas including lymphosarcoma, giant follicular lymphoma, Hodgkin's disease and non-Hodgkin's lymphomas.

Chlorambucil. Chlorambucil (also known as leukeran) is a bifunctional alkylating agent of the nitrogen mustard type that has been found active against selected human neoplastic diseases. Chlorambucil is known chemically as 4-[bis(2-chlorethyl)amino] benzenebutanoic acid.

Chlorambucil is available in tablet form for oral administration. It is rapidly and completely absorbed from the gastrointestinal tract. For example, after a single oral doses of about 0.6 mg/kg to about 1.2 mg/kg, peak plasma chlorambucil levels are reached within one hour and the terminal half-life of the parent drug is estimated at about 1.5 hours. About 0.1 mg/kg/day to about 0.2 mg/kg/day or about 36 mg/m²/day to about 6 mg/m²/day or alternatively about 0.4 mg/kg may be used for antineoplastic treatment. Chlorambucil is not curative by itself but may produce clinically useful palliation.

Cyclophosphamide. Cyclophosphamide is 2H-1,3,2-Oxazaphosphorin-2-amine, N,N-bis(2-chloroethyl)tetrahydro-, 2-oxide, monohydrate; termed Cytoxan available from Mead Johnson; and Neosar available from Adria. Cyclophosphamide is prepared by condensing 3-amino-1-propanol with N,N-bis(2-chlorethyl) phosphoramidic dichloride [(ClCH₂CH₂)₂N—POCl₂] in dioxane solution under the catalytic influence of triethylamine. The condensation is double, involving both the hydroxyl and the amino groups, thus effecting the cyclization.

Unlike other β-chloroethylamino alkylators, it does not cyclize readily to the active ethyleneimonium form until activated by hepatic enzymes. Thus, the substance is stable in the gastrointestinal tract, tolerated well and effective by the oral and parental routes and does not cause local vesication, necrosis, phlebitis or even pain.

Suitable oral doses for adults include, for example, about 1 mg/kg/day to about 5 mg/kg/day (usually in combination), depending upon gastrointestinal tolerance; or about 1 mg/kg/day to about 2 mg/kg/day; intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. In some aspects, a dose of about 250 mg/kg/day may be administered as an antineoplastic. Because of gastrointestinal adverse effects, the intravenous route is preferred for loading. During maintenance, a leukocyte count of about 3000/mm³ to 4000/mm³ usually is desired. The drug also sometimes is administered intramuscularly, by infiltration or into body cavities. It is available in dosage forms for injection of about 100 mg, about 200 mg and about 500 mg, and tablets of about 25 mg and about 50 mg.

Melphalan. Melphalan, also known as alkeran, L-phenylalanine mustard, phenylalanine mustard, L-PAM, or L-sarcolysin, is a phenylalanine derivative of nitrogen mustard. Melphalan is a bifunctional alkylating agent which is active against selective human neoplastic diseases. It is known chemically as 4-[bis(2-chloroethyl)amino]-L-phenylalanine.

Melphalan is the active L-isomer of the compound and was first synthesized in 1953 by Bergel and Stock; the D-isomer, known as medphalan, is less active against certain animal tumors, and the dose needed to produce effects on chromosomes is larger than that required with the L-isomer. The racemic (DL-) form is known as merphalan or sarcolysin. Melphalan is insoluble in water and has a pKa₁ of about 2.1. Melphalan is available in tablet form for oral administration and has been used to treat multiple myeloma. Available evidence suggests that about one third to one half of the patients with multiple myeloma show a favorable response to oral administration of the drug.

Melphalan has been used in the treatment of epithelial ovarian carcinoma. One commonly employed regimen for the treatment of ovarian carcinoma has been to administer melphalan at a dose of about 0.2 mg/kg daily for five days as a single course. Courses are repeated about every four to five weeks depending upon hematologic tolerance (Smith and Rutledge, 1975; Young et al., 1978). Alternatively in certain embodiments, the dose of melphalan used could be as low as about 0.05 mg/kg/day or as high as about 3 mg/kg/day or greater.

ii. Ethylenimenes and Methymelamines

An ethylenimene and/or a methylmelamine include, but are not limited to, hexamethylmelamine, used to treat ovary cancer; and thiotepa, which has been used to treat bladder, breast and ovary cancer.

iii. Alkyl Sulfonates

An alkyl sulfonate includes but is not limited to such drugs as busulfan, which has been used to treat chronic granulocytic leukemia.

Busulfan (also known as myleran) is a bifunctional alkylating agent. Busulfan is known chemically as 1,4-butanediol dimethanesulfonate. Busulfan is available in tablet form for oral administration, wherein for example, each scored tablet contains about 2 mg busulfan and the inactive ingredients magnesium stearate and sodium chloride.

Busulfan is indicated for the palliative treatment of chronic myclogenous (myeloid, myelocytic, granulocytic) leukemia. Although not curative, busulfan reduces the total granulocyte mass, relieves symptoms of the disease, and improves the clinical state of the patient. Approximately 90% of adults with previously untreated chronic myelogenous leukemia will obtain hematologic remission with regression or stabilization of organomegaly following the use of busulfan. Busulfan has been shown to be superior to splenic irradiation with respect to survival times and maintenance of hemoglobin levels, and to be equivalent to irradiation at controlling splenomegaly.

iv. Nitrosourea

Nitrosureas, like alkylating agents, inhibit DNA repair proteins. They are used to treat non-Hodgkin's lymphomas, multiple myeloma, malignant melanoma, in addition to brain tumors. A nitrosourea include but is not limited to a carmustine (BCNU), a lomustine (CCNU), a semustine (methyl-CCNU) or a streptozocin. Semustine has been used in such cancers as a primary brain tumor, a stomach or a colon cancer. Stroptozocin has been used to treat diseases such as a malignant pancreatic insulinoma or a malignalnt carcinoid. Streptozocin has beeen used to treat such cancers as a malignant melanoma, Hodgkin's disease and soft tissue sarcomas.

Carmustine. Carmustine (sterile carmustine) is one of the nitrosoureas used in the treatment of certain neoplastic diseases. It is 1,3 bis(2-chloroethyl)-1-nitrosourea. It is lyophilized pale yellow flakes or congealed mass with a molecular weight of 214.06. It is highly soluble in alcohol and lipids, and poorly soluble in water. Carmustine is administered by intravenous infusion after reconstitution as recommended Although it is generally agreed that carmustine alkylates DNA and RNA, it is not cross resistant with other alkylators. As with other nitrosoureas, it may also inhibit several key enzymatic processes by carbamoylation of amino acids in proteins.

Carmustine is indicated as palliative therapy as a single agent or in established combination therapy with other approved chemotherapeutic agents in brain tumors such as glioblastoma, brainstem glioma, medullobladyoma, astrocytoma, ependymoma, and metastatic brain tumors. Also it has been used in combination with prednisone to treat multiple myeloma. Carmustine has been used in treating such cancers as a multiple myeloma or a malignant melanoma. Carmustine has proved useful, in the treatment of Hodgkin's Disease and in non-Hodgkin's lymphomas, as secondary therapy in combination with other approved drugs in patients who relapse while being treated with primary therapy, or who fail to respond to primary therapy.

Sterile carmustine is commonly available in 100 mg single dose vials of lyophilized material. The recommended dose of carmustine as a single agent in previously untreated patients is about 150 mg/m² to about 200 mg/m² intravenously every 6 weeks. This may be given as a single dose or divided into daily injections such as about 75 mg/m² to about 100 mg/m² on 2 successive days. When carmustine is used in combination with other myelosuppressive drugs or in patients in whom bone marrow reserve is depleted, the doses should be adjusted accordingly. Doses subsequent to the initial dose should be adjusted according to the hematologic response of the patient to the preceding dose. It is of course understood that other doses may be used in the present invention, for example about 10 mg/m², about 20 mg/m², about 30 mg/m², about 40 mg/m², about 50 mg/m², about 60 mg/m², about 70 mg/m², about 80 mg/m², about 90 mg/m² to about 100 mg/m².

Lomustine. Lomustine is one of the nitrosoureas used in the treatment of certain neoplastic diseases. It is 1-(2-chloro-ethyl)-3-cyclohexyl-1 nitrosourea. It is a yellow powder with the empirical formula of C₉H₁₆ClN₃O₂ and a molecular weight of 233.71. Lomustine is soluble in 10% ethanol (about 0.05 mg/mL) and in absolute alcohol (about 70 mg/mL). Lomustine is relatively insoluble in water (less than about 0.05 mg/mL). It is relatively unionized at a physiological pH. Inactive ingredients in lomustine capsules are: magnesium stearate and mannitol.

Although it is generally agreed that lomustine alkylates DNA and RNA, it is not cross resistant with other alkylators. As with other nitrosoureas, it may also inhibit several key enzymatic processes by carbamoylation of amino acids in proteins.

Lomustine may be given orally. Following oral administration of radioactive lomustine at doses ranging from about 30 mg/m² to 100 mg/m², about half of the radioactivity given was excreted in the form of degradation products within 24 hours. The serum half-life of the metabolites ranges from about 16 hours to about 2 days. Tissue levels are comparable to plasma levels at 15 minutes after intravenous administration.

Lomustine has been shown to be useful as a single agent in addition to other treatment modalities, or in established combination therapy with other approved chemotherapeutic agents in both primary and metastatic brain tumors, in patients who have already received appropriate surgical and/or radiotherapeutic procedures. Lomustine has been used to treat such cancers as small-cell lung cancer. It has also proved effective in secondary therapy against Hodgkin's Disease in combination with other approved drugs in patients who relapse while being treated with primary therapy, or who fail to respond to primary therapy.

The recommended dose of lomustine in adults and children as a single agent in previously untreated patients is about 130 mg/m² as a single oral dose every 6 weeks. In individuals with compromised bone marrow function, the dose should be reduced to about 100 mg/m² every 6 weeks. When lomustine is used in combination with other myelosuppressive drugs, the doses should be adjusted accordingly. It is understood that other doses may be used for example, about 20 mg/m², about 30 mg/m², about 40 mg/m², about 50 mg/m², about 60 mg/m², about 70 mg/m², about 80 mg/m², about 90 mg/m², about 100 mg/m² to about 120 mg/m².

Triazine. A triazine include but is not limited to such drugs as a dacabazine (DTIC; dimethyltriazenoimidaz olecarboxamide), used in the treatment of such cancers as a malignant melanoma, Hodgkin's disease and a soft-tissue sarcoma.

b. Antimetabolites

Antimetabolites disrupt DNA and RNA synthesis. Unlike alkylating agents, they specifically influence the cell cycle during S phase. They have used to combat chronic leukemias in addition to tumors of breast, ovary and the gastrointestinal tract. Antimetabolites can be differentiated into various categories, such as folic acid analogs, pyrimidine analogs and purine analogs and related inhibitory compounds. Antimetabolites include but are not limited to, 5-fluorouracil (5-FU), cytarabine (Ara-C), fludarabine, gemcitabine, and methotrexate.

i. Folic Acid Analogs

Folic acid analogs include but are not limited to compounds such as methotrexate (amethopterin), which has been used in the treatment of cancers such as acute lymphocytic leukemia, choriocarcinoma, mycosis fungoides, breast, head and neck, lung and osteogenic sarcoma.

ii. Pyrimidine Analogs

Pyrimidine analogs include such compounds as cytarabine (cytosine arabinoside), 5-fluorouracil (fluouracil; 5-FU) and floxuridine (fluorode-oxyuridine; FudR). Cytarabine has been used in the treatment of cancers such as acute granulocytic leukemia and acute lymphocytic leukemias. Floxuridine and 5-fluorouracil have been used in the treatment of cancers such as breast, colon, stomach, pancreas, ovary, head and neck, urinary bladder and topical premalignant skin lesions. 5-Fluorouracil (5-FU) has the chemical name of 5-fluoro-2,4(1H,3H)-pyrimidinedione. Its mechanism of action is thought to be by blocking the methylation reaction of deoxyuridylic acid to thymidylic acid. Thus, 5-FU interferes with the synthesis of deoxyribonucleic acid (DNA) and to a lesser extent inhibits the formation of ribonucleic acid (RNA). Since DNA and RNA are essential for cell division and proliferation, it is thought that the effect of 5-FU is to create a thymidine deficiency leading to cell death. Thus, the effect of 5-FU is found in cells that rapidly divide, a characteristic of metastatic cancers.

iii. Purine Analogs and Related Inhibitors

Purine analogs and related compounds include, but are not limited to, mercaptopurine (6-mercaptopurine; 6-MP), thioguanine (6-thioguanine; TG) and pentostatin (2-deoxycoformycin). Mercaptopurine has been used in acute lymphocytic, acute granulocytic and chronic granulocytic leukemias. Thrioguanine has been used in the treatment of such cancers as acute granulocytic leukemia, acute lymphocytic leukemia and chronic lymphocytic leukemia. Pentostatin has been used in such cancers as hairy cell leukemias, mycosis fungoides and chronic lymphocytic leukemia.

c. Natural Products

Natural products generally refer to compounds originally isolated from a natural source, and identified has having a pharmacological activity. Such compounds, analogs and derivatives thereof may be, isolated from a natural source, chemically synthesized or recombinantly produced by any technique known to those of skill in the art. Natural products include such categories as mitotic inhibitors, antitumor antibiotics, enzymes and biological response modifiers.

i. Mitotic Inhibitors

Mitotic inhibitors include plant alkaloids and other natural agents that can inhibit either protein synthesis required for cell division or mitosis. They operate during a specific phase during the cell cycle. Mitotic inhibitors include, for example, docetaxel, etoposide (VP16), teniposide, paclitaxel, taxol, vinblastine, vincristine, and vinorelbine.

Epipodophyllotoxins. Epipodophyllotoxins include such compounds as teniposide and VP16. VP16 is also known as etoposide and is used primarily for treatment of testicular tumors, in combination with bleomycin and cisplatin, and in combination with cisplatin for small-cell carcinoma of the lung. Teniposide and VP16 are also active against cancers such as testis, other lung cancer, Hodgkin's disease, non-Hodgkin's lymphomas, acute granulocytic leukemia, acute nonlymphocytic leukemia, carcinoma of the breast, and Kaposi's sarcoma associated with acquired immunodeficiency syndrome (AIDS).

VP16 is available as a solution (e.g., 20 mg/ml) for intravenous administration and as 50 mg, liquid-filled capsules for oral use. For small-cell carcinoma of the lung, the intravenous dose (in combination therapy) is can be as much as about 100 mg/m² or as little as about 2 mg/m², routinely about 35 mg/m², daily for about 4 days, to about 50 mg/m², daily for about 5 days have also been used. When given orally, the dose should be doubled. Hence the doses for small cell lung carcinoma may be as high as about 200 mg/m² to about 250 mg/m². The intravenous dose for testicular cancer (in combination therapy) is about 50 mg/m² to about 100 mg/m² daily for about 5 days, or about 100 mg/m² on alternate days, for three doses. Cycles of therapy are usually repeated about every 3 to 4 weeks. The drug should be administered slowly (e.g., about 30 minutes to about 60 minutes) as an infusion in order to avoid hypotension and bronchospasm, which are probably due to the solvents used in the formulation.

Taxoids. Taxoids are a class of related compounds isolated from the bark of the ash tree, Taxus brevifolia. Taxoids include but are not limited to compounds such as docetaxel and paclitaxel.

Paclitaxel binds to tubulin (at a site distinct from that used by the vinca alkaloids) and promotes the assembly of microtubules. Paclitaxel is being evaluated clinically; it has activity against malignant melanoma and carcinoma of the ovary. In certain aspects, maximal doses are about 30 mg/m² per day for about 5 days or about 210 mg/m² to about 250 mg/m² given once about every 3 weeks.

Vinca Alkaloids. Vinca alkaloids are a type of plant alkaloid identified to have pharmaceutical activity. They include such compounds as vinblastine (VLB) and vincristine. Vinblastine is an example of a plant alkaloid that can be used for the treatment of cancer and precancer. When cells are incubated with vinblastine, dissolution of the microtubules occurs.

Unpredictable absorption has been reported after oral administration of vinblastine or vincristine. At the usual clinical doses the peak concentration of each drug in plasma is approximately 0.4 mM. Vinblastine and vincristine bind to plasma proteins. They are extensively concentrated in platelets and to a lesser extent in leukocytes and erythrocytes.

After intravenous injection, vinblastine has a multiphasic pattern of clearance from the plasma; after distribution, drug disappears from plasma with half-lives of approximately 1 and 20 hours. Vinblastine is metabolized in the liver to biologically activate derivative desacetylvinblastine. Approximately 15% of an administered dose is detected intact in the urine, and about 10% is recovered in the feces after biliary excretion. Doses should be reduced in patients with hepatic dysfunction. At least a 50% reduction in dosage is indicated if the concentration of bilirubin in plasma is greater than 3 mg/dl (about 50 mM).

Vinblastine sulfate is available in preparations for injection. When the drug is given intravenously; special precautions must be taken against subcutaneous extravasation, since this may cause painful irritation and ulceration. The drug should not be injected into an extremity with impaired circulation. After a single dose of 0.3 mg/kg of body weight, myelosuppression reaches its maximum in about 7 days to about 10 days. If a moderate level of leukopenia (approximately 3000 cells/mm³) is not attained, the weekly dose may be increased gradually by increments of about 0.05 mg/kg of body weight. In regimens designed to cure testicular cancer, vinblastine is used in doses of about 0.3 mg/kg about every 3 weeks irrespective of blood cell counts or toxicity.

An important clinical use of vinblastine is with bleomycin and cisplatin in the curative therapy of metastatic testicular tumors. Beneficial responses have been reported in various lymphomas, particularly Hodgkin's disease, where significant improvement may be noted in 50 to 90% of cases. The effectiveness of vinblastine in a high proportion of lymphomas is not diminished when the disease is refractory to alkylating agents. It is also active in Kaposi's sarcoma, testis cancer, neuroblastoma, and Letterer-Siwe disease (histiocytosis X), as well as in carcinoma of the breast and choriocarcinoma in women. Doses of about 0.1 mg/kg to about 0.3 mg/kg can be administered or about 1.5 mg/m² to about 2 mg/m² can also be administered. Alternatively, about 0.1 mg/m², about 0.12 mg/m², about 0.14 mg/m², about 0.15 mg/m², about 0.2 mg/m², about 0.25 mg/m², about 0.5 mg/m², about 1.0 mg/m², about 1.2 mg/m², about 1.4 mg/m², about 1.5 mg/m², about 2.0 mg/m², about 2.5 mg/m², about 5.0 mg/m², about 6 mg/m², about 8 mg/m², about 9 mg/m², about 10 mg/m², to about 20 mg/m², can be given.

Vincristine blocks mitosis and produces metaphase arrest. It seems likely that most of the biological activities of this drug can be explained by its ability to bind specifically to tubulin and to block the ability of protein to polymerize into microtubules. Through disruption of the microtubules of the mitotic apparatus, cell division is arrested in metaphase. The inability to segregate chromosomes correctly during mitosis presumably leads to cell death.

The relatively low toxicity of vincristine for normal marrow cells and epithelial cells make this agent unusual among anti-neoplastic drugs, and it is often included in combination with other myelosuppressive agents.

Unpredictable absorption has been reported after oral administration of vinblastine or vincristine. At the usual clinical doses the peak concentration of each drug in plasma is about 0.4 mM.

Vinblastine and vincristine bind to plasma proteins. They are extensively concentrated in platelets and to a lesser extent in leukocytes and erythrocytes. Vincristine has a multiphasic pattern of clearance from the plasma; the terminal half-life is about 24 hours. The drug is metabolized in the liver, but no biologically active derivatives have been identified. Doses should be reduced in patients with hepatic dysfunction. At least a 50% reduction in dosage is indicated if the concentration of bilirubin in plasma is greater than about 3 mg/dl (about 50 mM).

Vincristine sulfate is available as a solution (e.g., 1 mg/ml) for intravenous injection. Vincristine used together with corticosteroids is presently the treatment of choice to induce remissions in childhood leukemia; the optimal dosages for these drugs appear to be vincristine, intravenously, about 2 mg/m² of body-surface area, weekly; and prednisone, orally, about 40 mg/m², daily. Adult patients with Hodgkin's disease or non-Hodgkin's lymphomas usually receive vincristine as a part of a complex protocol. When used in the MOPP regimen, the recommended dose of vincristine is about 1.4 mg/m². High doses of vincristine seem to be tolerated better by children with leukemia than by adults, who may experience sever neurological toxicity. Administration of the drug more frequently than every 7 days or at higher doses seems to increase the toxic manifestations without proportional improvement in the response rate. Precautions should also be used to avoid extravasation during intravenous administration of vincristine. Vincristine (and vinblastine) can be infused into the arterial blood supply of tumors in doses several times larger than those that can be administered intravenously with comparable toxicity.

Vincristine has been effective in Hodgkin's disease and other lymphomas. Although it appears to be somewhat less beneficial than vinblastine when used alone in Hodgkin's disease, when used with mechlorethamine, prednisone, and procarbazine (the so-called MOPP regimen), it is the preferred treatment for the advanced stages (III and IV) of this disease. In non-Hodgkin's lymphomas, vincristine is an important agent, particularly when used with cyclophosphamide, bleomycin, doxorubicin, and prednisone. Vincristine is more useful than vinblastine in lymphocytic leukemia. Beneficial response have been reported in patients with a variety of other neoplasms, particularly Wilms' tumor, neuroblastoma, brain tumors, rhabdomyosarcoma, small cell lung, and carcinomas of the breast, bladder, and the male and female reproductive systems.

Doses of vincristine include about 0.01 mg/kg to about 0.03 mg/kg or about 0.4 mg/m² to about 1.4 mg/m² can be administered or about 1.5 mg/m² to about 2 mg/m² can also be administered. Alternatively, in certain embodiments, about 0.02 mg/m², about 0.05 mg/m², about 0.06 mg/m², about 0.07 mg/m², about 0.08 mg/m², about 0.1 mg/m², about 0.12 mg/m², about 0.14 mg/m², about 0.15 mg/m², about 0.2 mg/m², about 0.25 mg/m² can be given as a constant intravenous infusion.

Antitumor Antibiotics. Antitumor antibiotics have both antimicrobial and cytotoxic activity. These drugs also interfere with DNA by chemically inhibiting enzymes and mitosis or altering cellular membranes. These agents are not phase specific so they work in all phases of the cell cycle. Thus, they are widely used for a variety of cancers. Examples of antitumor antibiotics include, but are not limited to, bleomycin, dactinomycin, daunorubicin, doxorubicin (Adriamycin), plicamycin (mithramycin) and idarubicin. Widely used in clinical setting for the treatment of neoplasms these compounds generally are administered through intravenous bolus injections or orally.

Doxorubicin hydrochloride, 5,12-Naphthacenedione, (8s-cis)-10-[(3-amino-2,3,6-trideoxy-a-L-lyxo-hexopyranosyl)oxy]-7,8,9,10-tetrahydro-6,8, 11-trihydroxy-8-(hydroxyacetyl)-1-methoxy-hydrochloride (hydroxydaunorubicin hydrochloride, Adriamycin) is used in a wide antineoplastic spectrum. It binds to DNA and inhibits nucleic acid synthesis, inhibits mitosis and promotes chromosomal aberrations.

Administered alone, it is the drug of first choice for the treatment of thyroid adenoma and primary hepatocellular carcinoma. It is a component of 31 first-choice combinations for the treatment of diseases including ovarian, endometrial and breast tumors, bronchogenic oat-cell carcinoma, non-small cell lung carcinoma, stomach, genitourinary, thyroid, gastric adenocarcinoma, retinoblastoma, neuroblastoma, mycosis fungoides, pancreatic carcinoma, prostatic carcinoma, bladder carcinoma, myeloma, diffuse histiocytic lymphoma, Wilms' tumor, Hodgkin's disease, adrenal tumors, osteogenic sarcoma, soft tissue sarcoma, Ewing's sarcoma, rhabdomyosarcoma and acute lymphocytic leukemia. It is an alternative drug for the treatment of other diseases such as islet cell, cervical, testicular and adrenocortical cancers. It is also an immunosuppressant. Doxorubicin is absorbed poorly and is preferably administered intravenously. The pharmacokinetics are multicompartmental. Distribution phases have half-lives of 12 minutes and 3.3 hours. The elimination half-life is about 30 hours, with about 40% to about 50% secreted into the bile. Most of the remainder is metabolized in the liver, partly to an active metabolite (doxorubicinol), but a few percent is excreted into the urine. In the presence of liver impairment, the dose should be reduced.

In certain embodiments, appropriate intravenous doses are, adult, about 60 mg/m² to about 75 mg/m² at about 21-day intervals or about 25 mg/m² to about 30 mg/m² on each of 2 or 3 successive days repeated at about 3 week to about 4 week intervals or about 20 mg/m² once a week. The lowest dose should be used in elderly patients, when there is prior bone-marrow depression caused by prior chemotherapy or neoplastic marrow invasion, or when the drug is combined with other myelopoietic suppressant drugs. The dose should be reduced by about 50% if the serum bilirubin lies between about 1.2 mg/dL and about 3 mg/dL and by about 75% if above about 3 mg/dL. The lifetime total dose should not exceed about 550 mg/m² in patients with normal heart function and about 400 mg/m² in persons having received mediastinal irradiation. In certain embodiments, and alternative dose regiment may comprise about 30 mg/m² on each of 3 consecutive days, repeated about every 4 week. Exemplary doses may be about 10 mg/m², about 20 mg/m², about 30 mg/m², about 50 mg/m², about 100 mg/m², about 150 mg/m², about 175 mg/m², about 200 mg/m², about 225 mg/m², about 250 mg/m², about 275 mg/m², about 300 mg/m², about 350 mg/m², about 400 mg/m², about 425 mg/m², about 450 mg/m², about 475 mg/m², to about 500 mg/m².

Daunorubicin hydrochloride, 5,12-Naphthacenedione, (8S-cis)-8-acetyl-10-[(3-amino-2,3,6-trideoxy-a-L-lyxo-hexanopyranosyl)oxy]-7,8,9,10-tetrahydro-6,8,11-trihydroxy-10-methoxy-, hydrochloride; also termed cerubidine and available from Wyeth. Daunorubicin (daunomycin; rubidomycin) intercalates into DNA, blocks DAN-directed RNA polymerase and inhibits DNA synthesis. It can prevent cell division in doses that do not interfere with nucleic acid synthesis.

In combination with other drugs it is often included in the first-choice chemotherapy of diseases such as, for example, acute granulocytic leukemia, acute myelocytic leukemia in adults (for induction of remission), acute lymphocytic leukemia and the acute phase of chronic myclocytic leukemia. Oral absorption is poor, and it preferably given by other methods (e.g., intravenously). The half-life of distribution is 45 minutes and of elimination, about 19 hours. The half-life of its active metabolite, daunorubicinol, is about 27 hours. Daunorubicin is metabolized mostly in the liver and also secreted into the bile (about 40%). Dosage must be reduced in liver or renal insufficiencies.

Generally, suitable intravenous doses are (base equivalent): adult, younger than 60 years, about 45 mg/m²/day (about 30 mg/m² for patients older than 60 year.) for about 1 day, about 2 days or about 3 days about every 3 weeks or 4 weeks or about 0.8 mg/kg/day for about 3 days, about 4 days, about 5 days to about 6 days about every 3 weeks or about 4 weeks; no more than about 550 mg/m² should be given in a lifetime, except only about 450 mg/m² if there has been chest irradiation; children, about 25 mg/m² once a week unless the age is less than 2 years. or the body surface less than about 0.5 m, in which case the weight-based adult schedule is used. It is available in injectable dosage forms (base equivalent) of about 20 mg (as the base equivalent to about 21.4 mg of the hydrochloride). Exemplary doses may be about 10 mg/m², about 20 mg/m², about 30 mg/m², about 50 mg/m², about 100 mg/m², about 150 mg/m², about 175 mg/m², about 200 mg/m², about 225 mg/m², about 250 mg/m², about 275 mg/m², about 300 mg/m², about 350 mg/m², about 400 mg/m², about 425 mg/m², about 450 mg/m², about 475 mg/m², to about 500 mg/m².

Mitomycin (also known as mutamycin and/or mitomycin-C) is an antibiotic isolated from the broth of Streptomyces caespitosus which has been shown to have antitumor activity. The compound is heat stable, has a high melting point, and is freely soluble in organic solvents.

Mitomycin selectively inhibits the synthesis of deoxyribonucleic acid (DNA). The guanine and cytosine content correlates with the degree of mitomycin-induced cross-linking. At high concentrations of the drug, cellular RNA and protein synthesis are also suppressed. Mitomycin has been used in tumors such as stomach, cervix, colon, breast, pancreas, bladder and head and neck.

In humans, mitomycin is rapidly cleared from the serum after intravenous administration. Time required to reduce the serum concentration by about 50% after a 30 mg. bolus injection is 17 minutes. After injection of 30 mg, 20 mg, or 10 mg I.V., the maximal serum concentrations were 2.4 mg/mL, 1.7 mg/mL, and 0.52 mg/mL, respectively. Clearance is effected primarily by metabolism in the liver, but metabolism occurs in other tissues as well. The rate of clearance is inversely proportional to the maximal serum concentration because, it is thought, of saturation of the degradative pathways. Approximately 10% of a dose of mitomycin is excreted unchanged in the urine. Since metabolic pathways are saturated at relatively low doses, the percent of a dose excreted in urine increases with increasing dose. In children, excretion of intravenously administered mitomycin is similar.

Actinomycin D (Dactinomycin) [50-76-0]; C₆₂H₈₆N₁₂O₁₆ (1255.43) is an antineoplastic drug that inhibits DNA-dependent RNA polymerase. It is often a component of first-choice combinations for treatment of diseases such as, for example, choriocarcinoma, embryonal rhabdomyosarcoma, testicular tumor, Kaposi's sarcoma and Wilms' tumor. Tumors that fail to respond to systemic treatment sometimes respond to local perfusion. Dactinomycin potentiates radiotherapy. It is a secondary (efferent) immunosuppressive.

In certain specific aspects, actinomycin D is used in combination with agents such as, for example, primary surgery, radiotherapy, and other drugs, particularly vincristine and cyclophosphamide. Antineoplastic activity has also been noted in Ewing's tumor, Kaposi's sarcoma, and soft-tissue sarcomas. Dactinomycin can be effective in women with advanced cases of choriocarcinoma. It also produces consistent responses in combination with chlorambucil and methotrexate in patients with metastatic testicular carcinomas. A response may sometimes be observed in patients with Hodgkin's disease and non-Hodgkin's lymphomas. Dactinomycin has also been used to inhibit immunological responses, particularly the rejection of renal transplants.

Half of the dose is excreted intact into the bile and 10% into the urine; the half-life is about 36 hours. The drug does not pass the blood-brain barrier. Actinomycin D is supplied as a lyophilized powder (0/5 mg in each vial). The usual daily dose is about 10 mg/kg to about 15 mg/kg; this is given intravenously for about 5 days; if no manifestations of toxicity are encountered, additional courses may be given at intervals of about 3 weeks to about 4 weeks. Daily injections of about 100 mg to about 400 mg have been given to children for about 10 days to about 14 days; in other regimens, about 3 mg/kg to about 6 mg/kg, for a total of about 125 mg/kg, and weekly maintenance doses of about 7.5 mg/kg have been used. Although it is safer to administer the drug into the tubing of an intravenous infusion, direct intravenous injections have been given, with the precaution of discarding the needle used to withdraw the drug from the vial in order to avoid subcutaneous reaction. Exemplary doses may be about 100 mg/m², about 150 mg/m², about 175 mg/m², about 200 mg/m², about 225 mg/m², about 250 mg/m², about 275 mg/m², about 300 mg/m², about 350 mg/m², about 400 mg/m², about 425 mg/m², about 450 mg/m², about 475 mg/m², to about 500 mg/m².

Bleomycin is a mixture of cytotoxic glycopeptide antibiotics isolated from a strain of Streptomyces verticillus. Although the exact mechanism of action of bleomycin is unknown, available evidence would seem to indicate that the main mode of action is the inhibition of DNA synthesis with some evidence of lesser inhibition of RNA and protein synthesis.

In mice, high concentrations of bleomycin are found in the skin, lungs, kidneys, peritoneum, and lymphatics. Tumor cells of the skin and lungs have been found to have high concentrations of bleomycin in contrast to the low concentrations found in hematopoietic tissue. The low concentrations of bleomycin found in bone marrow may be related to high levels of bleomycin degradative enzymes found in that tissue.

In patients with a creatinine clearance of greater than about 35 mL per minute, the serum or plasma terminal elimination half-life of bleomycin is approximately 115 minutes. In patients with a creatinine clearance of less than about 35 mL per minute, the plasma or serum terminal elimination half-life increases exponentially as the creatinine clearance decreases. In humans, about 60% to about 70% of an administered dose is recovered in the urine as active bleomycin. In specific embodiments, bleomycin may be given by the intramuscular, intravenous, or subcutaneous routes. It is freely soluble in water. Because of the possibility of an anaphylactoid reaction, lymphoma patients should be treated with two units or less for the first two doses. If no acute reaction occurs, then the regular dosage schedule may be followed.

In certain aspects, bleomycin should be considered a palliative treatment. It has been shown to be useful in the management of the following neoplasms either as a single agent or in proven combinations with other approved chemotherapeutic agents in squamous cell carcinoma such as head and neck (including mouth, tongue, tonsil, nasopharynx, oropharynx, sinus, palate, lip, buccal mucosa, gingiva, epiglottis, larynx), esophagus, lung and genitourinary tract, Hodgkin's disease, non-Hodgkin's lymphoma, skin, penis, cervix, and vulva. It has also been used in the treatment of lymphomas and testicular carcinoma.

Improvement of Hodgkin's Disease and testicular tumors is prompt and noted within 2 weeks. If no improvement is seen by this time, improvement is unlikely. Squamous cell cancers respond more slowly, sometimes requiring as long as 3 weeks before any improvement is noted.

d. Miscellaneous Agents

Some chemotherapy agents do not qualify into the previous categories based on their activities. They include, but are not limited to, platinum coordination complexes, anthracenedione, substituted urea, methyl hydrazine derivative, adrenalcortical suppressant, amsacrine, L-asparaginase, and tretinoin. It is contemplated that they are included within the compositions and methods of the present invention for use in combination therapies.

i. Platinum Coordination Complexes

Platinum coordination complexes include such compounds as carboplatin and cisplatin (cis-DDP). Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes, such as for example, intravenous, subcutaneous, intratumoral or intraperitoneal injection. Cisplatin can be used alone or in combination with other agents, with efficacious doses used in clinical applications of about 15 mg/m² to about 20 mg/m² for 5 days every three weeks for a total of three courses being contemplated in certain embodiments. Doses may be, for example, about 0.50 mg/m², about 1.0 mg/m², about 1.50 mg/m², about 1.75 mg/m², about 2.0 mg/m², about 3.0 mg/m², about 4.0 mg/m², about 5.0 mg/m², to about 10 mg/m².

ii. Other Agents

An anthracenedione such as mitoxantrone has been used for treating acute granulocytic leukemia and breast cancer. A substituted urea such as hydroxyurea has been used in treating chronic granulocytic leukemia, polycythemia vera, essental thrombocytosis and malignant melanoma. A methyl hydrazine derivative such as procarbazine (N-methylhydrazine, MIH) has been used in the treatment of Hodgkin's disease. An adrenocortical suppressant such as mitotane has been used to treat adrenal cortex cancer, while aminoglutethimide has been used to treat Hodgkin's disease.

4. Toxins

Various toxins are also useful in the treatment of cancers. As part of the present invention, toxins such as ricin A-chain (Burbage, 1997), diphtheria toxin A (Massuda et al., 1997; Lidor, 1997), pertussis toxin A subunit, E. coli enterotoxin toxin A subunit, cholera toxin A subunit and Pseudomonas toxin c-terminal are suitable. It has demonstrated that transfection of a plasmid containing the fusion protein regulatable diphtheria toxin A chain gene was cytotoxic for cancer cells.

5. Liposomal Delivery Vehicles

In particular embodiments, the ST peptides of the present invention may be used in conjunction with a lipid delivery vehicle, often called liposomes. A “liposome” is a generic term encompassing a variety of single and multilamellar lipid vehicles formed by the generation of enclosed lipid bilayers or aggregates. Liposomes may be characterized as having vesicular structures with a bilayer membrane, generally comprising a phospholipid, and an inner medium that generally comprises an aqueous composition.

A multilamellar liposome has multiple lipid layers separated by aqueous medium. They form spontaneously when lipids comprising phospholipids are suspended in an excess of aqueous solution. The lipid components undergo self-rearrangement before the formation of closed structures and entrap water and dissolved solutes between the lipid bilayers (Ghosh and Bachhawat, 1991). Lipophilic molecules or molecules with lipophilic regions may also dissolve in or associate with the lipid bilayer.

A liposome used according to the present invention can be made by different methods, as would be known to one of ordinary skill in the art. For example, a phospholipid (Avanti Polar Lipids, Alabaster, Ala.), such as for example the neutral phospholipid dioleoylphosphatidylcholine (DOPC), is dissolved in tert-butanol. The lipid(s) is then mixed with the imexon and/or a derivative thereof, and/or other component(s). Tween 20 is added to the lipid mixture such that Tween 20 is about 5% of the composition's weight. Excess tert-butanol is added to this mixture such that the volume of tert-butanol is at least 95%. The mixture is vortexed, frozen in a dry ice/acetone bath and lyophilized overnight. The lyophilized preparation is stored at −20° C. and can be used up to three months. When required the lyophilized liposomes are reconstituted in 0.9% saline. The average diameter of the particles obtained using Tween 20 is about 0.7 to about 1.0 μm in diameter.

Alternatively, a liposome can be prepared by mixing lipids in a solvent in a container, e.g., a glass, pear-shaped flask. The container should have a volume ten-times greater than the volume of the expected suspension of liposomes. Using a rotary evaporator, the solvent is removed at approximately 40° C. under negative pressure. The solvent normally is removed within about 5 min. to 2 hours, depending on the desired volume of the liposomes. The composition can be dried further in a desiccator under vacuum. The dried lipids generally are discarded after about 1 week because of a tendency to deteriorate with time.

Dried lipids can be hydrated at approximately 25-50 mM phospholipid in sterile, pyrogen-free water by shaking until all the lipid film is resuspended. The aqueous liposomes can be then separated into aliquots, each placed in a vial, lyophilized and sealed under vacuum.

In other alternative methods, liposomes can be prepared in accordance with other known laboratory procedures (e.g., see Bangham et al., 1965; Gregoriadis, 1979; Deamer and Nichols, 1983; Szoka and Papahadjopoulos, 1978, each incorporated herein by reference in relevant part). These methods differ in their respective abilities to entrap aqueous material and their respective aqueous space-to-lipid ratios.

The dried lipids or lyophilized liposomes prepared as described above may be dehydrated and reconstituted in a solution of inhibitory peptide and diluted to an appropriate concentration with an suitable solvent, e.g., DPBS. The mixture is then vigorously shaken in a vortex mixer. Unencapsulated additional materials, such as agents including but not limited to hormones, drugs, nucleic acid constructs and the like, are removed by centrifugation at 29,000×g and the liposomal pellets washed. The washed liposomes are resuspended at an appropriate total phospholipid concentration, e.g., about 50-200 mM. The amount of additional material or active agent encapsulated can be determined in accordance with standard methods. After determination of the amount of additional material or active agent encapsulated in the liposome preparation, the liposomes may be diluted to appropriate concentrations and stored at 4° C. until use. A pharmaceutical composition comprising the liposomes will usually include a sterile, pharmaceutically acceptable carrier or diluent, such as water or saline solution.

The size of a liposome varies depending on the method of synthesis. Liposomes in the present invention can be a variety of sizes. In certain embodiments, the liposomes are small, e.g., less than about 100 nm, about 90 nm, about 80 nm, about 70 nm, about 60 nm, or less than about 50 nm in external diameter. In preparing such liposomes, any protocol described herein, or as would be known to one of ordinary skill in the art may be used. Additional non-limiting examples of preparing liposomes are described in U.S. Pat. Nos. 4,728,578, 4,728,575, 4,737,323, 4,533,254, 4,162,282, 4,310,505, and 4,921,706; International Applications PCT/US85/01161 and PCT/US89/05040; U.K. Patent Application GB 2193095 A; Mayer et al., 1986; Mayhew et al., 1984, each incorporated herein by reference).

A liposome suspended in an aqueous solution is generally in the shape of a spherical vesicle, having one or more concentric layers of lipid bilayer molecules. Each layer consists of a parallel array of molecules represented by the formula XY, wherein X is a hydrophilic moiety and Y is a hydrophobic moiety. In aqueous suspension, the concentric layers are arranged such that the hydrophilic moieties tend to remain in contact with an aqueous phase and the hydrophobic regions tend to self-associate. For example, when aqueous phases are present both within and without the liposome, the lipid molecules may form a bilayer, known as a lamella, of the arrangement XY-YX. Aggregates of lipids may form when the hydrophilic and hydrophobic parts of more than one lipid molecule become associated with each other. The size and shape of these aggregates will depend upon many different variables, such as the nature of the solvent and the presence of other compounds in the solution.

The production of lipid formulations often is accomplished by sonication or serial extrusion of liposomal mixtures after (I) reverse phase evaporation (II) dehydration-rehydration (III) detergent dialysis and (IV) thin film hydration. In one aspect, a contemplated method for preparing liposomes in certain embodiments is heating sonicating, and sequential extrusion of the lipids through filters or membranes of decreasing pore size, thereby resulting in the formation of small, stable liposome structures. This preparation produces liposomes only of appropriate and uniform size, which are structurally stable and produce maximal activity. Such techniques are well-known to those of skill in the art (see, for example Martin, 1990).

Numerous disease treatments are using lipid based gene transfer strategies to enhance conventional or establish novel therapies, in particular therapies for treating hyperproliferative diseases. Advances in liposome formulations have improved the efficiency of gene transfer in vivo (Templeton et al., 1997) and it is contemplated that liposomes are prepared by these methods. Alternate methods of preparing lipid-based formulations for nucleic acid delivery are described (WO 99/18933).

In another liposome formulation, an amphipathic vehicle called a solvent dilution microcarrier (SDMC) enables integration of particular molecules into the bi-layer of the lipid vehicle (U.S. Pat. No. 5,879,703). The SDMCs can be used to deliver lipopolysaccharides, polypeptides, nucleic acids and the like. Of course, any other methods of liposome preparation can be used by the skilled artisan to obtain a desired liposome formulation in the present invention.

Though liposomes may be used to deliver the radio- and chemotherapeutics discussed above, they find particular use in the delivery of gene therapy vectors, immunotherapy agents and hormonal therapy agents, each discussed further in the following pages.

A. Gene Therapy Vectors

Tumor cell resistance to agents, such as chemotherapeutic and radiotherapeutic agents, represents a major problem in clinical oncology. One goal of current cancer research is to find ways to improve the efficacy of one or more anti-cancer agents by combining such an agent with gene therapy. For example, the herpes simplex-thymidine kinase (HS-tK) gene, when delivered to brain tumors by a retroviral vector system, successfully induced susceptibility to the antiviral agent ganciclovir (Culver et al., 1992). In the context of the present invention, it is contemplated that gene therapy could be enhanced by specific cell targeting afforded by ST peptides, as discussed below.

i. Inducers of Cellular Proliferation

In one embodiment of the present invention, it is contemplated that antisense mRNA directed to a particular inducer of cellular proliferation is used to prevent expression of the inducer of cellular proliferation. The proteins that induce cellular proliferation further fall into various categories dependent on function. The commonality of all of these proteins is their ability to regulate cellular proliferation.

For example, a form of PDGF, the sis oncogene, is a secreted growth factor. Oncogenes rarely arise from genes encoding growth factors, and at the present, sis is the only known naturally-occurring oncogenic growth factor.

The proteins FMS, ErbA, ErbB and neu are growth factor receptors. Mutations to these receptors result in loss of regulatable function. For example, a point mutation affecting the transmembrane domain of the Neu receptor protein results in the neu oncogene. The erbA oncogene is derived from the intracellular receptor for thyroid hormone. The modified oncogenic ErbA receptor is believed to compete with the endogenous thyroid hormone receptor, causing uncontrolled growth.

The largest class of oncogenes includes the signal transducing proteins (e.g., Src, Abl and Ras). The protein Src is a cytoplasmic protein-tyrosine kinase, and its transformation from proto-oncogene to oncogene in some cases, results via mutations at tyrosine residue 527. In contrast, transformation of GTPase protein ras from proto-oncogene to oncogene, in one example, results from a valine to glycine mutation at amino acid 12 in the sequence, reducing ras GTPase activity.

Other proteins such as Jun, Fos and Myc are proteins that directly exert their effects on nuclear functions as transcription factors.

ii. Inhibitors of Cellular Proliferation

In certain embodiments, the restoration of the activity of an inhibitor of cellular proliferation through a genetic construct is contemplated. Tumor suppressor oncogenes function to inhibit excessive cellular proliferation. The inactivation of these genes destroys their inhibitory activity, resulting in unregulated proliferation. The tumor suppressors Rb, p53, p16 and C-CAM are described below.

High levels of mutant p53 have been found in many cells transformed by chemical carcinogenesis, ultraviolet radiation, and several viruses. The p53 gene is a frequent target of mutational inactivation in a wide variety of human tumors and is already documented to be the most frequently mutated gene in common human cancers. It is mutated in over 50% of human NSCLC (Hollstein et al., 1991) and in a wide spectrum of other tumors.

The p53 gene encodes a 393-amino acid phosphoprotein that can form complexes with host proteins such as large-T antigen and E1B. The protein is found in normal tissues and cells, but at concentrations which are minute by comparison with transformed cells or tumor tissue Wild-type p53 is recognized as an important growth regulator in many cell types. Missense mutations are common for the p53 gene and are essential for the transforming ability of the oncogene. A single genetic change prompted by point mutations can create carcinogenic p53. Unlike other oncogenes, however, p53 point mutations are known to occur in at least 30 distinct codons, often creating dominant alleles that produce shifts in cell phenotype without a reduction to homozygosity. Additionally, many of these dominant negative alleles appear to be tolerated in the organism and passed on in the germ line. Various mutant alleles appear to range from minimally dysfunctional to strongly penetrant, dominant negative alleles (Weinberg, 1991).

Another inhibitor of cellular proliferation is p16. The major transitions of the eukaryotic cell cycle are triggered by cyclin-dependent kinases, or CDK's. One CDK, cyclin-dependent kinase 4 (CDK4), regulates progression through the G₁. The activity of this enzyme may be to phosphorylate Rb at late G₁. The activity of CDK4 is controlled by an activating subunit, D-type cyclin, and by an inhibitory subunit, the p16^{INK4} has been biochemically characterized as a protein that specifically binds to and inhibits CDK4, and thus may regulate Rb phosphorylation (Serrano et al., 1993; Serrano et al., 1995). Since the p16^{INK4} protein is a CDK4 inhibitor (Serrano, 1993), deletion of this gene may increase the activity of CDK4, resulting in hyperphosphorylation of the Rb protein. p16 also is known to regulate the function of CDK6.

p16^{INK4} belongs to a newly described class of CDK-inhibitory proteins that also includes p16^{B}, p19, p21^{WAF1}, and p27^{KIP1} The p16^{INK4} gene maps to 9p21, a chromosome region frequently deleted in many tumor types. Homozygous deletions and mutations of the p16^{INK4} gene are frequent in human tumor cell lines. This evidence suggests that the p16^{INK4} gene is a tumor suppressor gene. This interpretation has been challenged, however, by the observation that the frequency of the p16^{INK4} gene alterations is much lower in primary uncultured tumors than in cultured cell lines (Caldas et al., 1994; Cheng et al., 1994; Hussussian et al., 1994; Kamb et al., 1994; Kamb et al, 1994; Okamoto et al., 1994; Nobori et al., 1995; Orlow et al., 1994; Arap et al., 1995). Restoration of wild-type p16^{INK4} function by transfection with a plasmid expression vector reduced colony formation by some human cancer cell lines (Okamoto, 1994; Arap, 1995).

Other genes that may be employed according to the present invention include Rb, APC, DCC, NF-1, NF-2, WT-1, MEN-I, MEN-II, zac1, p73, VHL, MMAC1/PTEN, DBCCR-1, FCC, rsk-3, p27, p27/p16 fusions, p21/p27 fusions, anti-thrombotic genes (e.g., COX-1, TFPI), PGS, Dp, E2F, ras, myc, neu, raf, erb, fms, trk, ret, gsp, hst, abl, E1A, p300, genes involved in angiogenesis (e.g., VEGF, FGF, thrombospondin, BAI-1, GDAIF, or their receptors) and MCC.

iii. Regulators of Programmed Cell Death

In certain embodiments, it is contemplated that genetic constructs that stimulate apoptosis will be used to promote the death of diseased or undesired tissue. Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl-2 protein now is recognized to be a member of a family of related proteins, which can be categorized as death agonists or death antagonists.

Subsequent to its discovery, it was shown that Bcl-2 acts to suppress cell death triggered by a variety of stimuli. Also, it now is apparent that there is a family of Bcl-2 cell death regulatory proteins which share in common structural and sequence homologies. These different family members have been shown to either possess similar functions to Bcl-2 (e.g., Bcl_{XL}, Bcl_{W}, Bcl_{S}, Mcl-1, A1, Bfl-1) or counteract Bcl-2 function and promote cell death (e.g., Bax, Bak, Bik, Bim, Bid, Bad, Harakiri).

B. Immunotherapy

An immunotherapeutic agent generally triggers immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. Various effector cells include cytotoxic T cells and NK cells.

i. Immune Stimulators

A specific aspect of immunotherapy is to use an immune stimulating molecule as an agent, or more preferably in conjunction with another agent, such as for example, a cytokines such as for example IL-2, IL-4, IL-12, GM-CSF, tumor necrosis factor; interferons alpha, beta, and gamma; F42K and other cytokine analogs; a chemokine such as for example MIP-1, MIP-1beta, MCP-1, RANTES, IL-8; or a growth factor such as for example FLT3 ligand.

One particular cytokine contemplated for use in the present invention is tumor necrosis factor. Tumor necrosis factor (TNF; Cachectin) is a glycoprotein that kills some kinds of cancer cells, activates cytokine production, activates macrophages and endothelial cells, promotes the production of collagen and collagenases, is an inflammatory mediator and also a mediator of septic shock, and promotes catabolism, fever and sleep. Some infectious agents cause tumor regression through the stimulation of TNF production. TNF can be quite toxic when used alone in effective doses, so that the optimal regimens probably will use it in lower doses in combination with other drugs. Its immunosuppressive actions are potentiated by gamma-interferon, so that the combination potentially is dangerous. A hybrid of TNF and interferon-α also has been found to possess anti-cancer activity.

Another cytokine specifically contemplate is interferon alpha. Interferon alpha has been used in treatment of hairy cell leukemia, Kaposi's sarcoma, melanoma, carcinoid, renal cell cancer, ovary cancer, bladder cancer, non-Hodgkin's lymphomas, mycosis fungoides, multiple myeloma, and chronic granulocytic leukemia.

ii. Active Immunotherapy

In active immunotherapy, an antigenic peptide, polypeptide or protein, or an autologous or allogenic tumor cell composition or “vaccine” is administered, generally with a distinct bacterial adjuvant (Ravindranath & Morton, 1991). In melanoma immunotherapy, those patients who elicit high IgM response often survive better than those who elicit no or low IgM antibodies. IgM antibodies are often transient antibodies and the exception to the rule appears to be anti-ganglioside or anticarbohydrate antibodies.

iii. Adoptive Immunotherapy

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg et al, 1988; 1989). To achieve this, one would administer to an animal, or human patient, an immunologically effective amount of activated lymphocytes in combination with an adjuvant-incorporated anigenic peptide composition as described herein. The activated lymphocytes will most preferably be the patient's own cells that were earlier isolated from a blood or tumor sample and activated (or “expanded”) in vitro. This form of immunotherapy has produced several cases of regression of melanoma and renal carcinoma, but the percentage of responders were few compared to those who did not respond.

C. Hormonal Therapy

Hormonal therapy may also be used in conjunction with the present invention and in combination with any other cancer therapy or agent(s). The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

i. Adrenocorticosteroids

Corticosteroid hormones are useful in treating some types of cancer (e.g., non-Hodgkin's lymphoma, acute and chronic lymphocytic leukemias, breast cancer, and multiple myeloma). Though these hormones have been used in the treatment of many non-cancer conditions, they are considered chemotherapy drugs when they are implemented to kill or slow the growth of cancer cells. Corticosteroid hormones can increase the effectiveness of other chemotherapy agents, and consequently, they are frequently used in combination treatments. Prednisone and dexamethasone are examples of corticosteroid hormones.

ii. Other Hormones and Antagonists

Progestins such as hydroxyprogesterone caproate, medroxyprogesterone acetate, and megestrol acetate have been used in cancers of the endometrium and breast. Estrogens such as diethylstilbestrol and ethinyl estradiol have been used in cancers such as breast and prostate. Antiestrogens such as tamoxifen have been used in cancers such as breast. Androgens such as testosterone propionate and fluoxymesterone have also been used in treating breast cancer. Antiandrogens such as flutamide have been used in the treatment of prostate cancer. Gonadotropin-releasing hormone analogs such as leuprolide have been used in treating prostate cancer.

VI. Combination Therapies

In order to increase the effectiveness of a given cancer therapy, it may be desirable to combine that therapy with another anti-cancer agent or therapeutic regimens. An “anti-cancer” agent or therapeutic regimen is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. A suitable secondary agent/therapy includes chemotherapy, radiation therapy, surgery, hormonal therapy, gene therapy, immunotherapy or other method.

Generally, these other compositions/methods are provided in a combined amount effective to kill or inhibit proliferation of the cell. This process may involve contacting the cells with the ST peptide-related agent and the second agent or therapy at the same time. This may be achieved by contacting the cell with a single composition or pharmacological formulation that includes two agents, or by contacting the cell with two distinct compositions or formulations, at the same time, wherein one composition includes the ST peptide-related agent, and the other includes the second agent.

Alternatively, the secondary therapy may precede or follow the ST peptide-related treatment by intervals ranging from minutes to weeks. In embodiments where the secondary agent and ST peptide agent are applied separately to the cell, one would generally ensure that a significant period of time did not expire between the time of each delivery, such that the two therapies would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, however, where several d (2, 3, 4, 5, 6 or 7) to several wk (1, 2, 3, 4, 5, 6, 7 or 8) lapse between the respective administrations.

Various combinations may be employed, where the ST peptide-related therapy is “A” and the secondary agent is “B”:

A/B/A B/A/B B/B/A A/A/B A/B/B B/A/A A/B/B/B
B/A/B/B B/B/B/A B/B/A/B A/A/B/B A/B/A/B A/B/B/A
B/B/A/A B/A/B/A B/A/A/B A/A/A/B B/A/A/A A/B/A/A
A/A/B/A

It is expected that the treatment cycles would be repeated as necessary, or may be used continuously for indefinite periods of time.

VII. Pharmaceutical Compositions

Pharmaceutical aqueous compositions of the present invention comprise an effective amount of one ST peptide conjugates dissolved or dispersed in a pharmaceutically acceptable carrier or aqueous medium. The phrases “pharmaceutically or pharmacologically acceptable” refer to molecular entities and compositions that do not produce an adverse, allergic or other untoward reaction when administered to a human. As used herein, “pharmaceutically acceptable carrier” includes any and all solvents, dispersion media, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The actual dosage amount of a composition of the present invention administered to a patient can be determined by physical and physiological factors such as body weight, severity of condition, idiopathy of the patient and on the route of administration. With these considerations in mind, the dosage of a lipid composition for a particular subject and/or course of treatment can readily be determined.

The compositions of the present invention can be administered intravenously, intradermally, intraarterially, intraperitoneally, intralesionally, intracranially, intraarticularly, intraprostaticaly, intrapleurally, intratracheally, intranasally, intravitreally, intravaginally, rectally, topically, intratumorally, intramuscularly, intraperitoneally, subcutaneously, intravesicularlly, mucosally, intrapericardially, orally, topically, locally using aerosol, injection, infusion, continuous infusion, localized perfusion bathing target cells directly or via a catheter or lavage. Typically, such compositions are prepared as injectables, either as liquid solutions or suspensions; solid forms suitable for preparing solutions or suspensions upon the addition of a liquid prior to injection can also be prepared; and the preparations can also be emulsified. The compositions will be sterile, be fluid to the extent that easy syringability exists, stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

Although it is most preferred that compositions of the present invnetion be prepared in sterile water containing other non-active ingredients, made suitable for injection, solutions of such active ingredients can also be prepared in water suitably mixed with a surfactant, such as hydroxypropylcellulose, if desired. Dispersions can also be prepared in liquid polyethylene glycols, and mixtures thereof and in oils. The carrier can also be a solvent or dispersion medium containing, for example, water, ethanol, polyol (for example, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants.

The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Upon formulation, solutions will be administered in a manner compatible with the dosage formulation and in such amount as is therapeutically effective. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. Some variation in dosage will necessarily occur depending on the condition of the subject being treated. The person responsible for administration will, in any event, determine the appropriate dose for the individual subject.

VIII. Kits

Any of the compositions described herein may be comprised in a kit. In a non-limiting example, an ST peptide or analogue thereof may be comprised in a kit. The kits will thus comprise, in suitable container means, an ST peptide, with optional additional agents of the present invention, such as linking reagents or diagnostic and/or therapeutic agents.

The kits may comprise a suitably aliquoted ST peptide or analogues thereof, whether conjugated or not. The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the containers in close confinement for commercial sale. Such means may include injection or blow-molded plastic containers into which the desired vials are retained.

### IX. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

All solvents were either ACS certified or HPLC grade solvents were obtained from Fisher Scientific and used as received. The fmoc-Phe-Wang resin and fmoc-protected amino acids were purchased from Calbiochem-Novabiochem Corp (San Diego, Calif.) and the other peptide reagents from Applied Biosystems, Inc (Foster City, Calif.). DOTA-tris(t-butyl ester) was purchased from Macrocyclics (Dallas, Tex.) and fmoc-6-aminohexanoic acid from Advanced ChemTech (Louisville, Ky.). All other reagents were purchased from Aldrich Chemical Company. ¹¹¹InCl₃ was obtained from Mallinckrodt Medical, Inc (St. Louis, Mo.) as a 0.05N HCl solution. ¹²⁵I-Tyr⁵-6-Ahx-Phe¹⁹-STₕ and ¹²⁵I-Tyr⁵-Phe¹⁹-STₕ were synthesized according to the previously published procedure. Human cancer cells were obtained from American Type Culture Collection (ATCC) and maintained and grown for use in these studies in the University of Missouri Cell and Immunology Core facilities. Electrospray mass spectral analyses were performed by Synpep Corporation (Dublin, Calif.).

High performance liquid chromatography (HPLC). High performance liquid chromatography (HPLC) analyses were performed on a Waters 600E system equipped with Varian 2550 variable absorption detector, Packard Radiometic 150TR flow scintillation analyzer, sodium iodide crystal radiometric detector, Eppendorf TC-50 column temperature controller and Hewlett Packard HP3395 integrators. HPLC solvents consisted of H₂O containing 0.1% trifluoroacetic acid (Solvent A) and acetonitrile containing 0.1% trifluoroacetic acid (Solvent B). Conditions: A Phenomenex Jupiter C-18 (5 μm, 300 A°, 4.6×250 mm) column was used with a flow rate of 1.5 ml/min. The column temperature was maintained at 35° C. Gradient I begins with a solvent composition of 95% A and 5% B followed by a linear gradient to 30% A:70% B in 25 min, after which the column is re-equilibrated. Gradient II begins with a solvent composition of 80% A and 20% B followed by a linear gradient to 70% A:30% B in 30 min, after which the column is re-equilibrated.

DOTA-Phe¹⁹-STₕ. Linear peptide DOTA-[Cys^{6,11}, Cys(Acm)^{7,15}, Cys(tBu)^{10,18}]-Phe¹⁹-STₕ synthesis was carried out on a Perkin Elmer—Applied Biosystems Model 432 automated peptide synthesizer employing traditional fmoc chemistry with HBTU activation of carboxyl groups on the reactant with the N-terminal amino group on the growing peptide anchored via the C-terminus to the resin. Fmoc-Phe-Wang resin (25 μmol), fmoc-protected amino acid with appropriate side-chain protections (75 μmol) and DOTA-tris(t-butyl ester) (75 μmol) were used for the synthesis. The final product was cleaved by a standard procedure using a cocktail containing thioanisol, water, ethanedithiol and trifluoroacetic acid in a ratio of 2:1:1:36 and precipitated into methyl-t-butyl ether and dried. Yield of the crude peptide was 90% (60 mg). First folding: DOTA-[Cys^{6,11}, Cys(Acm)^{7,15}, Cys(tBu)^{10,18}]-Phe¹⁹-STₕ (63 mg, 22.6 μmol) was dissolved in water (110 ml) and the pH of the solution was adjusted to 8.6 using 0.1M NH₄OH (3.5 ml). To this solution was added dropwise 2,2′-dithiodipyridine (2-PDS) (10 mg, 45.5 μmol) in methanol (15 ml) while stirring at room temperature. The stirring was continued for an hour and the reaction mixture was concentrated and filtered. The filtrate was purified on the HPLC (tᵣ=17.6 min, Gradient I) and lyophilized to give pure DOTA-[Cys(Acm)^{7,15}, Cys(tBu)^{10,18}]-Phe¹⁹-STₕ as a white powder in a yield of 29% (18.3 mg). Electrospray MS calcd. m/z for C₁₀₉H₆₈N₂₈O₃₈S₆ [M+H]⁺: 2670.0; found: 2670.0. Second folding: DOTA-[Cys(Acm)^{7,15}, Cys(tBu)^{10,18}]-Phe¹⁹-STₕ (18 mg, 6.5 μmol) was dissolved in 80% aqueous methanol (7 ml) and 1M HCl (40 μl, 2.0 equiv) was added. To this solution was added dropwise iodine (40 mg, 157 μmol) dissolved in methanol (0.5 ml) while stirring at room temperature. The stirring was continued for 30 minutes and then 1M ascorbic acid (0.5 ml) was added to reduce the excess iodine. The reaction mixture was concentrated, purified on the HPLC (tᵣ=18.6 min, Gradient I) and lyophilized to give pure DOTA-[Cys(tBu)^{10,18}]-Phe¹⁹-STₕ as a white powder in a yield of 62% (10.2 mg). Electrospray MS calcd. m/z for C₁₀₃H₁₅₆N₂₆O₃₆S₆ [M+H]⁺: 2526.0; found: 2526.0. Third folding: To a solution of DOTA-[Cys(tBu)^{10,18}]-Phe¹⁹-STₕ (10 mg, 4.0 μmol) in TFA (5 ml) was added PhS(O)Ph (12 mg, 15 equiv) and thioanisole (80 μl, 160 equiv). To this solution was added CH₃SiCl₃ (110 μl, 22 equiv) while stirring at room temperature. The stirring was continued for 45 minutes and then the reaction mixture was added to methyl-t-butyl ether (40 ml) and extracted with water (2×10 ml). The aqueous solution was neutralized with 10% NH₄OH, concentrated, purified on the HPLC (tᵣ=20.0 min, Gradient II) and lyophilized to give pure DOTA-Phe¹⁹-STₕ as a white powder in a yield of 6% (0.6 mg). Electrospray MS calcd. m/z for C₉₅H₁₃₈N₂₆O₃₆S₆ [M+H]⁺: 2411.8; found: 2412.0.

DOTA-6-Ahx-Phe¹⁹-STₕ. The same synthetic procedure described to produce DOTA-Phe¹⁹-STₕ was used to prepare the DOTA-6-Ahx-Phe¹⁹-STₕ analog except that the fmoc-6-aminohexanoic acid was added in the reaction sequence. Overall yield (starting from resin): 1.5%; Electrospray MS calcd. m/z for C₁₀₁H₁₄₉N₂₇O₃₇S₆ [M+H]⁺: 2524.9; found: 2525.0.

Phe¹⁹-STₕ. The same synthetic procedure described to produce DOTA-Phe¹⁹-STₕ was used to prepare the Phe¹⁹-STₕ analog except that the DOTA-tris(t-butyl ester) was deleted from the reaction sequence. Overall yield (starting from resin): 1.6%; Electrospray MS calcd. m/z for C₇₉H₁₁₂N₂₂O₂₉S₆ [M+H]⁺: 2025.6; found: 2025.6.

Indium metallation. A solution of DOTA-Phe¹⁹-STₕ or DOTA-6-Ahx-Phe¹⁹-STₕ (0.5 mg) in 0.2M tetramethylammonium acetate (0.5 ml) was added to indium chloride (1.0 mg). The pH of the reaction mixture was adjusted to 5.8. The reaction mixture was incubated for 1 hour at 80° C. The resultant In-DOTA-STₕ conjugate was purified by reversed-phase HPLC (Gradient II). Electrospray MS calcd. m/z for C₉₅H₁₃₅N₂₆O₃₆S₆In (In-DOTA-Phe¹⁹-STₕ) [M+H]⁺: 2523.7; found: 2524.0.

¹¹¹In labeling. An aliquot of ¹¹¹InCl₃ (0.5-2.5 mCi, 1.85-9.25 MBq, 50 μl) was added to a solution of DOTA-Phe¹⁹-STₕ (50 μg) or DOTA-6-Ahx-Phe¹⁹-STₕ (50 μg) in 0.2M tetramethylammonium acetate (400 μl). The pH of the reaction mixture was adjusted to 5.8. The reaction mixture was incubated for 1 hour at 80° C. An aliquot of 0.002M EDTA (50 μl) was added to the reaction mixture to complex the unreacted ¹¹¹In⁺³. The resultant ¹¹¹In-DOTA-Phe¹⁹-STₕ or ¹¹¹In-DOTA-6-Ahx-Phe¹⁹-STₕ obtained as single products and purified by HPLC. The ¹¹¹In-DOTA-Phe¹⁹-STₕ or ¹¹¹In-DOTA-6-Ahx-Phe¹⁹-STₕ eluted approximately 2 minutes before the non-metallated DOTA-Phe¹⁹-STₕ or DOTA-6-Ahx-Phe¹⁹-STₕ conjugate (Gradient II) enabling collection of the high-specific activity, NCA ¹¹¹In-DOTA-Phe¹⁹-STₕ or ¹¹¹In-DOTA-6-Ahx-Phe¹⁹-STₕ conjugate. The ¹¹¹In-DOTA-Phe¹⁹-STₕ or ¹¹¹In-DOTA-6-Ahx-Phe¹⁹-STₕ were concentrated by passing through a 3M Empore C-18 HD high performance extraction disk (7 mm/3 ml) cartridge and eluting with 33% ethanol in 0.1M NaH₂PO₄ buffer (400 μl). The concentrated fraction were diluted with 0.1M NaH₂PO₄ buffer (2.3 ml, pH-7) to make the final concentration of ethanol in the solution <5%.

In Vitro Competitive Cell Binding Assay. The IC₅₀ values of both metallated and non-metallated STₕ conjugates were determined in human cancer cells by a competitive displacement cell binding assay using ¹²⁵I-STₕ (¹²⁵I-Tyr⁵-Phe¹⁹-STₕ or ¹²⁵I-Tyr⁵-6-Ahx-Phe¹⁹-STₕ). Briefly 3×10⁶ cells suspended in DMEM/F-12 media containing 14.4 mM MES and 2% BSA, pH-5.5, were incubated at 37° C. for 1 hr in presence of approximately 20,000 cpm ¹²⁵I-STₕ and increasing concentration of STₕ conjugates. After the incubation, the reaction medium was aspirated and cells were washed three times with media. The radioactivity bound to the cells was counted in a Packard Riastar gamma counting system. The ¹²⁵I-STₕ bound to cells was plotted vs. increasing concentrations of STₕ conjugate to determine the respective IC₅₀ values (Table 3). For statistical considerations, three separate in vitro cell binding experiments with each conjugate were performed in duplicate.

Scatchard Analysis. Scatchard analysis was performed in human cancer cells by a receptor-binding assay using ¹²⁵I-STₕ and 6-Ahx-Phe¹⁹-STₕ or Phe¹⁹-STₕ. Briefly 3.0×10⁶ cells suspended in DMEM/F-12 media containing 14.4 mM MES and 2% BSA, pH-5.5, were incubated at 37° C. for 1 hr in presence of approximately 25,000 cpm ¹²⁵I-STₕ and increasing concentration of 6-Ahx-Phe¹⁹-STₕ or Phe¹⁹-STₕ. After the incubation, the reaction medium was aspirated and cells were washed three times with media. The radioactivity bound to the cells was counted in a Packard Riastar gamma counting system. The experiment was performed in duplicate and average values were used for the calculations. The total bound peptide (B, both radioactive and non-radioactive), was obtained by multiplying the fraction of bound labeled peptide with the total concentration of peptide (both radioactive and non-radioactive). The total free peptide (F, both radioactive and non-radioactive) was obtained by substracting the total bound peptide from the total concentration of peptide. The non-specific binding was neglected in the calculations as it was <3%. The ratio of concentration of total bound and total free peptide (B/F) was plotted vs. total bound peptide (B) to determine the K_{d} (−1/slope) and Bₘₐₓ (X-intercept) values. The number of receptors per cell was calculated from the Bₘₐₓ (Table 4).

In vivo iodistribution studies. Four- to 5-week old female ICR SCID (severely compromised immunodeficient) outbred mice were obtained from Taconic (Germantown, N.Y.). The mice were housed five animals per cage in sterile micro isolator cages in a temperature- and humidity-controlled room with a 12-hour light/12-hour dark schedule. The animals were fed autoclaved rodent chow (Ralston Purina Company, St. Louis, Mo.) and water ad libitum. Animals were housed one week prior to inoculation of tumor cells and anesthetized for injections with isoflurane (Baxter Healthcare Corp., Deerfield, Ill.) at a rate of 2.5% with 0.4L oxygen through a non-rebreathing anesthesia vaporizer.

Human breast cancer MB231 and T47D cells were injected on the bilateral subcutaneous (s.c.) flank with ˜5×10⁶ cells in a suspension of 100 μl normal sterile saline per injection site. MB231 and T47D cells were allowed to grow in vivo two to three weeks post inoculation developing tumors ranging in sizes from 0.02-1.30 grams. The biodistribution and uptake of ¹¹¹In-DOTA-Phe¹⁹-STₕ or ¹¹¹In-DOTA-6-Ahx-Phe¹⁹-STₕ in tumor bearing SCID mice was studied. The mice (average weight, 25 g) were injected with aliquots (50-100 μl) of the radiolabeled peptide solution (55-75 kBq) in each animal via the tail vein. Tissues, organs and tumors were excised from the animals sacrificed at 1 hr, 4 hrs and 24 hrs p.i. For blocking studies, an access of non-radioactive 6-Ahx-Phe¹⁹-STₕ (100 μg) was also injected along with the radiolabeled peptide solution. The radioactivity was measured in a NaI counter and the percent-injected dose per organ and the percent-injected dose per gram tissue were calculated (Tables 5 and 6). Animal studies were conducted in accordance with the highest standards of care as outlined in the NIH guide for Care and Use of Laboratory Animals and the Policy and Procedures for Animal Research at the Harry S. Truman Memorial VA Hospital and according to approved protocols.

TABLE 3
In vitro IC₅₀ (nM) values measured from competitive binding assay with STₕ analogs vs. ¹²⁵I-Tyr⁵-6-Ahx-Phe¹⁹-STₕ or
¹²⁵I-Tyr⁵-Phe¹⁹-STₕ in different cancer cell line.

| | Breast | | | | Pancreatic | | | Lung | Ovarian | Prostate | Melanoma |
|---|---|---|---|---|---|---|---|---|---|---|---|
| | T47D | MB-231 | MB-468 | MCF-7 | CFPAC-1 | AR42J | CAPAN-1 | H69 | OVCAR-3 | PC-3 | A375 |
| STₕ analog | (Human) | (Human) | (Human) | (Human) | (Human) | (Rat) | (Human) | (Human) | (Human) | (Human) | (Human) |
| Phe¹⁹-STₕ | 3.0 ± 1.7 | 5.2 ± 1.3 | 2.8 | — | — | — | — | 4.5 ± 1.7 | 3.2 | — | — |
| 6-Ahx-Phe¹⁹- | 5.6 ± 0.9 | 5.2 ± 1.5 | — | 4.6 ± 2.8 | 4.4 ± 1.5 | — | 4.5 | — | — | 3.1 | 6.4 ± 2.9 |
| STₕ | | | | | | | | | | | |
| DOTA-6-Ahx- | 0.5 | 3.9 | — | 14.1 | 2.8 | — | — | — | — | 7.0 | 1.5 |
| Phe¹⁹-STₕ | | | | | | | | | | | |
| In-DOTA- | 8.9 ± 2.2 | 10.0 | 10.6 | — | 12.7 ± 3.4 | 13.2 ± 8.2 | — | 7.0 | — | — | — |
| Phe¹⁹-STₕ | | | | | | | | | | | |

TABLE 4
Scatchard analysis of STₕ analogs in different human cancer cell lines.

| | No. of | |
|---|---|---|
| | receptors per | |
| Cell Line | cell | K_{d} (nM) |
| Breast | | |
| T-47D | 41,758 | 4.4 |
| MB-231 | 112,786 | 4.0 |
| MB-468 | 42,588 | 3.1 |
| Pancreatic | | |
| CFPAC-1 | 242,094 | 6.9 |
| Lung | | |
| H-69 | 33,456 | 6.4 |
| Ovarian | | |
| OVCAR-3 | 13,386 | 3.2 |

TABLE 5
¹¹¹In-DOTA-6-Ahx-Phe¹⁹-STₕ biodistribution (Avg % ID/gm, n = 5)
in MB-231 tumor bearing SCID mice after 1 hr, 4 hrs & 24 hrs
post-injection.

| | | 1 hour (n = 4) | | 24 hours |
|---|---|---|---|---|
| Tissue | 1 hour | (Blocking) | 4 hours | (n = 4) |
| Blood | 0.57 ± 0.13 | 0.85 ± 0.31 | 0.04 ± 0.05 | 0.01 ± 0.02 |
| Heart | 0.14 ± 0.03 | 0.24 ± 0.11 | 0.06 ± 0.06 | 0.03 ± 0.03 |
| Lung | 0.31 ± 0.04 | 0.49 ± 0.12 | 0.08 ± 0.05 | 0.01 ± 0.02 |
| Liver | 0.20 ± 0.03 | 0.33 ± 0.06 | 0.09 ± 0.02 | 0.05 ± 0.02 |
| Spleen | 0.15 ± 0.10 | 0.12 ± 0.14 | 0.05 ± 0.05 | 0.02 ± 0.04 |
| Stomach | 0.31 ± 0.33 | 2.18 ± 3.69 | 0.03 ± 0.01 | 0.09 ± 0.09 |
| Large | 0.37 ± 0.05 | 0.17 ± 0.03 | 0.66 ± 0.52 | 1.50 ± 2.59 |
| Intestine | | | | |
| Small | 0.99 ± 0.18 | 0.85 ± 1.19 | 0.28 ± 0.07 | 0.21 ± 0.13 |
| Intestine | | | | |
| Kidney | 4.98 ± 0.67 | 12.80 ± 2.01 | 4.96 ± 0.50 | 2.94 ± 0.42 |
| Muscle | 0.10 ± 0.08 | 0.10 ± 0.08 | 0.04 ± 0.05 | 0.02 ± 0.04 |
| Pancreas | 0.13 ± 0.03 | 0.21 ± 0.04 | 0.05 ± 0.02 | 0.03 ± 0.02 |
| Tumor | 0.70 ± 0.16 | 0.84 ± 0.30 | 0.24 ± 0.12 | 0.13 ± 0.16 |
| Urine | 89.4 ± 2.9 | 87.4 ± 4.5 | 95.8 ± 1.4 | 82.5 ± 20.3 |
| (Avg % ID) | | | | |
| Feces | — | — | — | 14.2 ± 17.3 |
| (Avg % ID) | | | | |

TABLE 6
¹¹¹In-DOTA-Phe¹⁹-STₕ biodistribution (Avg % ID/gm, n = 4) in T47D
tumor bearing SCID mice after 1 hr, 4 hrs & 24 hrs post-injection.

| | | 1 hour | | |
|---|---|---|---|---|
| Tissue | 1 hour | (Blocking) | 4 hours | 24 hours |
| Blood | 0.82 ± 0.34 | 0.57 ± 0.19 | 0.01 ± 0.01 | 0.02 ± 0.01 |
| Heart | 0.15 ± 0.11 | 0.13 ± 0.10 | 0.00 ± 0.00 | 0.08 ± 0.08 |
| Lung | 0.56 ± 0.19 | 0.45 ± 0.16 | 0.09 ± 0.06 | 0.06 ± 0.03 |
| Liver | 0.26 ± 0.12 | 0.19 ± 0.04 | 0.06 ± 0.01 | 0.03 ± 0.01 |
| Spleen | 0.19 ± 0.16 | 0.12 ± 0.07 | 0.13 ± 0.15 | 0.12 ± 0.08 |
| Stomach | 0.53 ± 0.47 | 0.61 ± 0.64 | 0.17 ± 0.06 | 0.02 ± 0.01 |
| Large | 0.57 ± 0.21 | 0.17 ± 0.06 | 1.74 ± 1.04 | 0.10 ± 0.03 |
| Intestine | | | | |
| Small | 1.37 ± 0.55 | 0.25 ± 0.10 | 0.44 ± 0.07 | 0.09 ± 0.02 |
| Intestine | | | | |
| Kidney | 4.70 ± 1.40 | 5.88 ± 1.47 | 2.02 ± 0.35 | 1.04 ± 0.33 |
| Muscle | 0.19 ± 0.08 | 0.18 ± 0.08 | 0.06 ± 0.04 | 0.04 ± 0.03 |
| Pancreas | 0.21 ± 0.09 | 0.16 ± 0.07 | 0.04 ± 0.05 | 0.02 ± 0.03 |
| Tumor | 0.67 ± 0.23 | 0.43 ± 0.20 | 0.17 ± 0.16 | 0.14 ± 0.12 |
| Urine | 86.4 ± 5.0 | 93.1 ± 1.5 | 96.1 ± 1.0 | 97.8 ± 0.7 |
| (Avg % ID) | | | | |
| Feces | — | — | — | 1.4 ± 0.7 |
| (Avg % ID) | | | | |

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### X. REFERENCES

The following references, to the extent that they provide exemplary procedural or other details supplementary to those set forth herein, are specifically incorporated herein by reference.

U.S. Pat. No. 4,162,282

U.S. Pat. No. 4,310,505

U.S. Pat. No. 4,472,509

U.S. Pat. No. 4,533,254

U.S. Pat. No. 4,728,575

U.S. Pat. No. 4,728,578

U.S. Pat. No. 4,737,323

U.S. Pat. No. 4,921,706

U.S. Pat. No. 4,938,948

U.S. Pat. No. 5,021,236

U.S. Pat. No. 5,401,511

U.S. Pat. No. 5,518,888

U.S. Pat. No. 5,603,872

U.S. Pat. No. 5,879,703

U.S. Pat. No. 5,889,155

Carrithers et al., Gastroenterology, 107(6):1653-61, 1994.

“The Proteins,” Vol. 11, 3d Ed., 105-237, Neurath et al., Eds., Academic Press, New York, N.Y., 1976.

Arap et al., Cancer Res., 55(6):1351-1354, 1995.

Bakhshi et al., Cell, 41(3):899-906, 1985.

Bangham et al., J. Mol. Biol., 13:238, 1965.

Bodanszky et al., J. Antibiot, 29(5):549-53, 1976.

Burbage et al., Leuk. Res., 21(7):681-690, 1997.

Caldas et al., Nat. Genet., 8(1):27-32, 1994.

Carrithers et al., Dis. Colon Rectum, February;39(2):171-81, 1996.

Cheng et al., Cancer Res., 54(21):5547-5551, 1994.

Cleary and Sklar, Proc. Nat'l Acad. Sci. USA, (21):7439-7443, 1985.

Cleary et al., J. Exp. Med., 164(1):315-320, 1986.

Culver et al., Science, 256(5063):1550-1552, 1992.

Deamer and Nichols, Proc Natl Acad Sci USA, 80(1):165-8, 1983.

Forte et al, Endocrinology, 140(4):1800-1806, 1999.

Ghosh and Bachhawat, In: Liver Diseases, Targeted Diagnosis and Therapy Using Specific Receptors and Ligands. Wu et al., eds., Marcel Dekker, New York, pp. 87-104, 1991.

Gregoriadis, Drug carriers in biology and medicine, 1979.

Hollstein et al., Science, 253(5015):49-53, 1991.

Hussussian et al., Nat. Genet., 8(1):15-21, 1994.

Kamb et al., Nat. Genet., 8(1):23-2, 1994.

Klaassen, In: The Pharmacological Basis of Therapeutics, Goodman and Gilman, Eds., Pergamon Press, 8th Ed., pp. 49-61, 1990.

Lidor et al., Am. J. Obstet. Gynecol., 177(3):579-585, 1997.

Martin, J. Biol. Chem., 265(34):20946-20951, 1990.

Massuda et al., Proc. Nat'l Acad. Sci. USA, 94(26):14701-14706, 1997.

Mayer et al., Biochim Biophys Acta, 858(1):161-8, 1986.

Mayhew et al., Biochim Biophys Acta, 775(2):169-74, 1984.

McOmie, Protective Groups in Organic Chemistry, Plenum Press, New York, N.Y., 1973.

Merrifield, J. Am. Chem. Soc., 15:2149-2154, 1963.

Nobori et al., Nature, 368(6473):753-756, 1994.

Okamoto et al., Proc. Nat'l Acad. Sci. USA, 1(23): 11045-11049, 1994.

Orlow et al., Cancer Res., 54(11):2848-2851, 1994.

PCT App. No. PCT/US85/01161

PCT App. No. PCT/US89/05040

PCT Appl. WO 99/18933

Peptide Synthesis, John Wiley & Sons, 2d Ed.; Kent & Clark-Lewis In: Synthetic Peptides in Biology and Medicine, Alitalo et al. (Eds.), 295-358, Science Publishers, (Amsterdam, 1985);

Ravindranath and Morton, Intern. Rev. Immunol., 7: 303-329, 1991.

Remington's Pharmaceutical Sciences, 18th Ed. Mack Printing Company, pp. 1289-1329, 1990.

Rosenberg et al., Ann. Surg. 210(4):474-548, 1989

Rosenberg et al., N. Engl. J Med., 319:1676, 1988.

Semrad, Gastroenterology, 113(3):1036-8, 1997.

Serrano et al., Nature, 366:704-707, 1993.

Serrano et al., Science, 267(5195):249-252, 1995.

Smith and Rutledge, Nat'l Cancer Inst. Monogr., 42:141-143, 1975.

Stuart & Young, Solid Phase Peptide Synthelia, Pierce Chemical Company, Rockford, Ill., 1984.

Szoka and Papahadjopoulos, Proc. Nat'l Acad. Sci. USA, 75:4194-4198, 1978.

Templeton et al., Nat. Biotechnol. 15(7):647-652, 1997.

Tsujimoto and Croce, Proc. Nat'l Acad. Sci. USA 83(14):5214-5218, 1986.

Tsujimoto et al., Science, 228(4706):1440-1443, 1985.

U.K. App. No. GB 2193095A

Weinberg, Science, 254(5035):1138-1146, 1991.

Young et al., N. Engl. J Med. 7:299(23):1261-1266, 1978.

What is claimed is:

1. A method for targeting an agent to a breast cancer cell, a prostate cancer cell, a pancreatic cancer cell or a melanoma cancer cell comprising bringing said cancer cell into contact with a peptide-agent complex, wherein said peptide comprises an ST motif that binds to breast cancer cells, prostate cancer cells, pancreatic cancer cells or melanoma cancer cells.

2. The method of claim 1, wherein said agent is a diagnostic agent.

3. The method of claim 2, wherein said diagnostic agent is a radiolabel, a chemilluminescent label, a fluorescent label, a magnetic spin resonance label, or a dye.

4. The method of claim 3, wherein the diagnostic agent is a radiolabel selected from the group consisting of astatine²¹¹, ⁵¹chromium, ³⁶chlorine, ⁵⁷cobalt, ⁵⁸cobalt, copper⁶⁷, ¹⁵²europium, gallium⁶⁷, iodine¹²³, iodine¹²⁵, iodine¹³¹, indium¹¹¹, ⁵⁹⁻iron, ³²phosphorus, rhenium¹⁸⁶, rhenium¹⁸⁸, ⁷⁵selenium, ³⁵sulphur, technicium^{99m}, yttrium⁹⁰, lutetium¹⁷⁷, samarium¹⁵³, holmium¹⁶⁶, and actinium²²⁵.

5. The method of claim 1, wherein said agent is a therapeutic agent.

6. The method of claim 5, wherein said therapeutic agent is a chemotherapeutic agent, a radiotherapeutic agent, a toxin, a cytokine or a nucleic acid construct.

7. The method of claim 1, wherein said ST motif is an STₕ motif.

8. The method of claim 7, wherein said STₕ motif comprises a Y—Rb₍₆₋₁₈₎—X, wherein Y is a tail region comprising a linear segment of 0-10 amino acid residues, Rb₍₆₋₁₈₎ is a receptor binding region, and X it Tyr or Phe.

9. The method of claim 8, wherein said tail region comprises Asn-Ser-Ser-Asn-Tyr.

10. The method of claim 8, wherein X is Tyr.

11. The method of claim 8, wherein X is Phe.

12. The method of claim 8, wherein said Rb₍₆₋₁₈₎ comprises Cys-Cys-Glu-Leu-Cys-Cys-Asn-Pro-Ala-Cys-Thr-Gly-Cys.

13. The method of claim 1, wherein said complex further comprises a linking moiety that connects said agent and said peptide.

14. The method of claim 13, wherein said linking moiety is linked to said ST peptide through the N-terminal amine.

15. The method of claim 1, wherein said cancer cell is located in a subject.

16. The method of claim 15, wherein is said subject is a human.

17. The method of claim 15, wherein said complex is delivered local or regional to said cancer cell.

18. The method of claim 15, wherein said complex is delivered systemically.

19. The method of claim 1, wherein said cancer cell is a breast cancer cell.

20. The method of claim 1, wherein said cancer cell is a prostate cancer cell.

21. The method of claim 1, wherein said cancer cell is a pancreatic cancer cell.

22. The method of claim 1, wherein said cancer cell is a melanoma cancer cell.

23. A method for diagnosing breast cancer, prostate cancer, pancreatic cancer or melanoma in a subject comprising:
(a) administering to said subject a peptide-diagnostic agent complex, wherein said peptide comprises an ST motif, wherein said ST motif binds to breast cancer cells, prostate cancer cells, pancreatic cancer cells or melanoma cancer cells; and
(b) assessing the amount and/or localization in said subject, of the diagnostic agent.

24. The method of claim 23, wherein said diagnostic agent is a radiolabel, a chemilluminescent label, a fluorescent label, a magnetic spin resonance label, or a dye.

25. The method of claim 23, wherein the diagnostic agent is a radiolabel selected from the group consisting of astatine²¹¹, ⁵¹chromium, ³⁶chlorine, ⁵⁸cobalt, ⁵⁸cobalt, copper⁶⁷, ¹⁵²europium, gallium⁶⁷, iodine¹²³, iodine¹²⁵, iodine¹³¹, indium¹¹¹, ⁵⁹⁻iron, ³²phosphorus, rhenium¹⁸⁶, rhenium¹⁸⁸, ⁷⁵selenium, ³⁵sulphur, technicium^{99m}, yttrium⁹⁰, lutetium¹⁷⁷, samarium¹⁵³, holmium¹⁶⁶, and actinium²²⁵.

26. The method of claim 23, wherein said ST motif is an STₕ motif.

27. The method of claim 26, wherein said STₕ motif comprises a Y—Rb₍₆₋₁₈₎—X, wherein Y is a tail region comprising a linear segment of 0-10 amino acid residues, Rb₍₆₋₁₈₎ is a receptor binding region, and X it Tyr or Phe.

28. The method of claim 27, wherein said tail region comprises Asn-Ser-Ser-Asn-Tyr.

29. The method of claim 27, wherein X is Tyr.

30. The method of claim 27, wherein X is Phe.

31. The method of claim 27, wherein said Rb₍₆₋₁₈) comprises Cys-Cys-Glu-Leu-Cys-Cys-Asn-Pro-Ala-Cys-Thr-Gly-Cys.

32. The method of claim 23, wherein said complex further comprises a linking moiety that connects said agent and said peptide.

33. The method of claim 32, wherein said linking moiety is linked to said ST peptide through the N-terminal amine.

34. The method of claim 23, wherein said complex is delivered local or regional to a tumor.

35. The method of claim 23, wherein said complex is delivered systemically.

36. The method of claim 23, wherein said cancer is breast cancer.

37. The method of claim 23, wherein said cancer is prostate cancer.

38. The method of claim 23, wherein said cancer is pancreatic cancer.

39. The method of claim 23, wherein said cancer is melanoma.

40. The method of claim 23, wherein said patient has not been previously diagnosed with cancer.

41. The method of claim 23, wherein said patient has been previously diagnosed with cancer.

42. The method of claim 41, wherein said patient has previously received a cancer therapy.

43. The method of claim 23, wherein said patient is at elevated risk for one or more of breast cancer, prostate cancer, pancreatic cancer or melanoma.

44. The method of claim 23, wherein assessing comprises organ or whole body imaging.

45. A method for treating breast cancer, prostate cancer, pancreatic cancer or melanoma in a subject in need thereof comprising administering to said subject a peptide-therapeutic agent complex, wherein said peptide comprises an ST motif and binds to breast cancer cells, prostate cancer cells, pancreatic cancer cells or melanoma cancer cells.

46. The method of claim 45, wherein said therapeutic agent is a chemotherapeutic agent, a radiotherapeutic agent, a toxin, a cytokine or a nucleic acid construct.

47. The method of claim 46, wherein the therapeutic agent is a radiolabel selected from the group consisting of astatine²¹¹, ⁵¹chromium, ³⁶chlorine, ⁵⁷cobalt, ⁵⁸cobalt, copper⁶⁷, ¹⁵²europium, gallium⁶⁷, iodine¹²³, iodine¹²⁵, iodine¹³¹, indium¹¹¹, ⁵⁹⁻iron, ³²phosphorus, rhenium¹⁸⁶, rhenium¹⁸⁸, ⁷⁵selenium, ³⁵sulphur, technicium^{99m}, yttrium⁹⁰, lutetium¹⁷⁷, samarium¹⁵³, holmium¹⁶⁶, and actinium^{225.}

48. The method of claim 45, wherein said ST motif is an STₕ motif.

49. The method of claim 48, wherein said STₕ motif comprises a Y—Rb₍₆₋₁₈₎—X, wherein Y is a tail region comprising a linear segment of 0-10 amino acid residues, Rb₍₆₋₁₈₎ is a receptor binding region, and X it Tyr or Phe.

50. The method of claim 49, wherein said tail region comprises Asn-Ser-Ser-Asn-Tyr.

51. The method of claim 49, wherein X is Tyr.

52. The method of claim 49, wherein X is Phe.

53. The method of claim 49, wherein said Rb₍₆₋₁₈₎ comprises Cys-Cys-Glu-Leu-Cys-Cys-Asn-Pro-Ala-Cys-Thr-Gly-Cys.

54. The method of claim 45, wherein said complex further comprises a linking moiety that connects said agent and said peptide.

55. The method of claim 54, wherein said linking moiety is linked to said ST peptide through the N-terminal amine.

56. The method of claim 45, wherein said cancer is breast cancer.

57. The method of claim 45, wherein said cancer is prostate cancer.

58. The method of claim 45, wherein said cancer is pancreatic cancer.

59. The method of claim 45, wherein said cancer is melanoma.

60. The method of claim 45, wherein said complex is administered more than once.

61. The method of claim 45, wherein said complex is delivered local or regional to a tumor.

62. The method of claim 45, wherein said complex is delivered systemically.

63. The method of claim 45, further comprising administering a second distinct cancer therapy.

64. The method of claim 63, wherein said second cancer therapy is radiotherapy, chemotherapy, immunotherapy or surgery.

65. A method for rendering an unresectable breast, prostate, pancreatic or melanoma tumor resectable comprising administering to a subject having said tumor a peptide-therapeutic agent complex, wherein said peptide comprises an ST motif that binds to breast cancer cells, prostate cancer cells, pancreatic cancer cells or melanoma cancer cells.

66. A method for treating metastatic breast cancer, prostate cancer, pancreatic cancer or melanoma comprising administering to a subject in need thereof a peptide-therapeutic agent complex, wherein said peptide comprises an ST motif that binds to breast cancer cells, prostate cancer cells, pancreatic cancer cells or melanoma cancer cells.

67. A method for preventing recurrent breast cancer, prostate cancer, pancreatic cancer or melanoma comprising administering to a subject having been successfully treated for breast cancer, prostate cancer, pancreatic cancer or melanoma a peptide-therapeutic agent complex, wherein said peptide comprises an ST motif that binds to breast cancer cells, prostate cancer cells, pancreatic cancer cells or melanoma cancer cells.

68. A method for identifying tumor binding peptides comprising:
(a) providing a breast cancer cell, a prostate cancer cell, a pancreatic cancer cell or a melanoma cell;
(b) contacting said cell, in the presence of a candidate peptide, with a labeled, tumor-binding ST peptide that binds to breast cancer cells, prostate cancer cells, pancreatic cancer cells or melanoma cancer cells;
(c) measuring the association of label with said cell, as compared to the association of label with said cell in the absence of said candidate peptide; and
(d) measuring binding of said candidate peptide to ST peptide, wherein a decrease in association of label with said cell, and the absence of candidate peptide binding to ST peptide, indicates that said candidate peptide is competing with ST peptide for tumor cell binding.

69. The method of claim 68, further comprising labeling said candidate peptide, incubating said labeled candidate peptide with said cell, and measuring the association of label with said cell.
